(19) United States
(12) Patent Application Publication
(10) Pub. No.: US 2005/0112122 A1
(43) Pub. Date: May 26, 2005

(54) Generation of hematopoietic chimerism and induction of central tolerance

(76) Inventors: Dale L. Greiner, Hubbardston, MA (US); John Mordes, Newton, MA (US); Aldo Rossini, Sudbury, MA (US); Edward Seung, Medford, MA (US)

Correspondence Address: FISH & RICHARDSON PC, 225 FRANKLIN ST, BOSTON, MA, 02110, US

(21) Appl. No.: 10/933,933

(22) Filed: Sep. 2, 2004

Related U.S. Application Data
(60) Provisional application No. 60/499,418, filed on Sep. 2, 2003.

(51) Int. Cl. A61K 39/395

(52) U.S. Cl. 424/144.1; 424/93.7

(57) ABSTRACT

The invention relates to the methods for producing hematopoietic chimerism and central tolerance by peripheral tolerance induction without myeloablative conditioning.

### CROSS-REFERENCE TO RELATED APPLICATION

This application claims priority from U.S. provisional patent application Ser. No. 60/499,418, filed Sep. 2, 2003, which is incorporated herein by reference in its entirety.

### FEDERALLY SPONSORED RESEARCH OR DEVELOPMENT

This invention was made with Government support under Grant No. AI42669 from the National Institutes of Health, an institutional Diabetes Endocrinology Research Center (DERC) Grant No. DK52530 from the National Institutes of Health, and Grant No. DK53006 jointly funded by the National Institutes of Health and the Juvenile Diabetes Research Foundation. The Government has certain rights in the invention.

### TECHNICAL FIELD

This invention relates to methods for the generation of hematopoietic chimerism and the induction of central tolerance.

### BACKGROUND

Allogeneic stem cell transplantation has significant potential for the treatment of malignancy (Appelbaum, Nature, 411:385-389, 2001), autoimmunity (Leykin et al., Transplant. Proc., 33:120, 2001; Slavin et al., Cancer Chemother. Pharmacol., 48 Suppl. 1:S79-S84, 2001; Solsky and Wallace, Best Pract. Res. Clin. Rheumatol., 16:293-312, 2002; and Openshaw et al., Biol. Blood Marrow Transplant., 8:233-248, 2002), and genetic disorders (Yang and Hill, Pediatr. Infect. Dis. J., 20:889-900, 2001; and Poulsom et al., J. Pathol., 197:441-456, 2002). It can also be used to facilitate gene therapy (Bordignon and Roncarolo, Nat. Immunol., 3:318-321, 2002; Emery et al., Int. J. Hematol., 75:228-236, 2002; Park et al., Gene Ther., 9:613-624, 2002; Desnick and Astrin, Br. J. Haematol., 117:779-795, 2002; and Bielorai et al., Isr. Med. Assoc. J., 4:648-652, 2002) and solid organ transplantation (Rossini et al., Physiol. Rev., 79:99-141, 1999; and Sorli et al., Graft, 1:71-81, 1998). Realization of that potential has been difficult, however, due to significant patient safety issues (Locatelli et al., Exp. Hem., 28:479-489, 2000).

Achieving allogeneic hematopoietic chimerism currently requires preparative conditioning with immunosuppression and at least partial myeloablation (Locatelli et al., 2000, supra). The conditioning required to establish stem cell engraftment is toxic, and even in partially ablated recipients, stem cell transplantation almost invariably leads to some degree of graft-versus-host disease (GVHD) (Rossini et al., Physiol. Rev., 79:99-141, 1999; Sorli et al., 1998, supra; Giralt et al., Blood, 89:4531-4536, 1997; Anderlini et al., Bone Marrow Transplant., 26:615-620, 2000; and Sykes, Immunity, 14:417-424, 2001). GVHD generally requires treatment with immunosuppressive drugs, which themselves have many toxic side effects (Laederach-Hofmann and Bunzel, Gen. Hosp. Psychiatry, 22:412-424, 2000). Both the conditioning regimen and immunosuppressive medications pose short-term risks of infection and longer-term risks of malignancy (Soulillou and Giral, Transplantation, 72:S89-S93, 2001).

To avoid both lethal conditioning and GVHD, strategies based on costimulation blockade have been developed. The combination of sub-lethal host conditioning and costimulation blockade has been shown to lead to the generation of allogeneic hematopoietic chimerism in mice (Sykes, 2001, supra; Forman et al., J. Immunol., 168:6047-6056. 2002; Seung et al., Blood, 95:2175-2182, 2000; Wekerle et al., J. Immunol., 166:2311-2316, 2001; Wekerle et al., J. Exp. Med., 187:2037-2044, 1998; Adams et al., J. Immunol., 167:1103-1111, 2001; Taylor et al., Blood, 98:467-474, 2001; and Durham et al., J. Immunol., 165:1-4, 2000). Approaches include sub-lethal irradiation plus CTLA4-Ig and/or anti-CD154 monoclonal antibody (monoclonal antibodies are also referred to herein as “mAb”) with or without peripheral T cell depletion (Seung et al., 2000, supra; Wekerle et al., 2001, supra; and Taylor et al., 2001, supra); anti-CD154 mAb plus drug-induced myeloablation (Adams et al., 2001, supra; and Kean et al., Blood, 99:1840-1849, 2002); and injection of supraphysiological doses of bone marrow over an extended time in combination with anti-CD 154 mAb without host conditioning (Durham et al., J. Immunol., 165:1-4, 2000; and Wekerle et al., Nat. Med., 6:464-469, 2000).

Hematopoietic chimerism generated by these methods involves intrathymic deletion of host Vβ CD4⁺ T cells reactive to donor superantigens presented by donor MHC class II I-E antigens. This result suggests that a state of central tolerance has been induced (Sykes, 2001, supra; and Wekerle and Sykes, Annu. Rev. Med., 52:353-370, 2001). Pre-existing peripheral host Vβ donor-reactive CD4⁺ T cells appear to die over time through both Fas-dependent and independent mechanisms (Wekerle et al., 2001, supra). A single trial of allogeneic stem cell transplantation for the treatment of leukemia based on ex vivo blockade of B7-mediated costimulation reportedly showed promising results (Guinan et al., N. Engl. J. Med., 340:1704-1714, 1999).

In the field of organ transplantation, the therapeutic potential of allogeneic hematopoietic chimerism resides in its ability to generate central tolerance, which is the most robust state of donor-specific transplantation tolerance known (Rossini et al., Physiol. Rev. 79:99-141, 1999; Sykes, 2001, supra; Adams et al., Philos. Trans. R. Soc. Lond. [Biol.], 356:703-705, 2001).

### SUMMARY

The present invention is based, at least in part, on the discovery that hematopoietic chimerism and durable central tolerance can be achieved in an allogeneic or xenogeneic transplant recipient by inducing peripheral tolerance, typically without any, or only minimal, myeloablative conditioning. In some aspects, the methods include inducing hematopoietic chimerism and central tolerance by administering to the recipient (a) a priming transfusion including allogeneic or xenogeneic cells, e.g., cells that express donor or third-party antigens (i.e., alloantigens) and that have at least one ligand on the surface that interacts with a receptor on the surface of a recipient T cell that mediates contact-dependent helper-effector function, e.g., CD154; (b) an antagonist of the receptor that inhibits interaction of the ligand with the receptor, e.g., a CD154 antagonist such as an anti-CD154 or -CD40 antibody; and (c) a hematopoietic stem cell transplant, e.g., a bone marrow transplant, e.g., a low-dose bone marrow transplant, thus inducing hematopoietic chimerism and central donor-specific tolerance. In some embodiments, the methods further include implanting a tissue or organ graft into the recipient. In some embodiments, the methods include administering to the recipient an anti-CD122 mAb in addition to or in place of a priming transfusion.

Peripheral tolerance protocols, in the absence of bone marrow engraftment, lead to transient deletion of peripheral, but not intrathymic, alloreactive CD8⁺ T cells. Previous studies of skin allografts on mice treated with a donor-specific transfusion (DST) and anti-CD154 mAb have shown that graft survival is greatly prolonged, but seldom permanent unless the recipient has been thymectomized (Markees et al., J. Clin. Invest., 101:2446-2455, 1998). The inference has been that the failure of graft maintenance is due to the release of new alloreactive thymic emigrants into the periphery (Iwakoshi et al., J. Immunol., 167:6623-6630, 2001). As described herein, the generation of hematopoietic chimerism, using the present methods, in effect “thymectomizes” recipients in a donor-specific manner, producing a durable, long-lasting, central tolerance, providing an essential complement to peripheral tolerance induction if allografts are to be truly durable.

In one aspect, the invention includes methods for inducing hematopoietic chimerism and central tolerance in a transplant recipient. The methods include administering to the recipient a priming transfusion including allogeneic or xenogeneic cells, wherein the cells have on their surface a ligand that interacts with a receptor on a surface of a recipient T cell that mediates contact-dependent helper-effector function; administering to the recipient one or more doses of a receptor antagonist that inhibits interaction of the ligand with the receptor, e.g., a CD154 antagonist such as an anti-CD154 antibody or an antigen-binding fragment thereof, or an anti-CD40 antibody or an antigen-binding fragment thereof; and administering to the recipient a hematopoietic stem cell transplant, e.g., a bone marrow transplant, e.g., a low-dose bone marrow transplant. In some embodiments, the methods include implanting into the recipient a tissue or organ, e.g., concurrently with or after administering the hematopoietic stem cell transplant. In some embodiments, the methods include administering to the recipient one or more doses of a CD122 antagonist, e.g., an anti-CD122 antibody or antigen-binding fragment thereof.

In another aspect, the invention includes other methods for inducing hematopoietic chimerism and central tolerance in a transplant recipient. The methods include administering to the recipient a CD122 antagonist, e.g., an anti-CD122 antibody or antigen-binding fragment thereof; administering to the recipient one or more doses of a CD154 antagonist such as an anti-CD154 antibody or an antigen-binding fragment thereof, or an anti-CD40 antibody or an antigen-binding fragment thereof; and administering to the recipient a hematopoietic stem cell transplant, e.g., a bone marrow transplant, e.g., a low-dose bone marrow transplant. In some embodiments, the methods include administering to the recipient a priming transfusion including allogeneic or xenogeneic cells, wherein the cells have on their surface a ligand that interacts with a receptor on a surface of a recipient T cell that mediates contact-dependent helper-effector function. In some embodiments, the methods include implanting into the recipient a tissue or organ, e.g., concurrently with or after administering the hematopoietic stem cell transplant.

In another aspect, the invention provides methods for implanting a tissue or organ into a transplant recipient. The methods include administering to the recipient a priming transfusion including allogeneic or xenogeneic cells, wherein the cells have on their surface a ligand that interacts with a receptor on a surface of a recipient T cell that mediates contact-dependent helper-effector function; administering to the recipient one or more doses of a receptor antagonist that inhibits interaction of the ligand with said receptor, e.g., a CD154 antagonist such as an anti-CD154 antibody or an antigen-binding fragment thereof, or an anti-CD40 antibody or an antigen-binding fragment thereof; administering to the recipient a hematopoietic stem cell transplant, e.g., a bone marrow transplant, e.g., a low-dose bone marrow transplant; and implanting into the recipient a tissue or organ, e.g., concurrently with or after administering the hematopoietic stem cell transplant. Alternatively, the method can include, in addition to or in place of a priming transfusion, administering to the recipient a CD122 antagonist, e.g., an anti-CD122 antibody or antigen-binding fragment thereof.

In some embodiments, the allogeneic or xenogeneic cells of the priming transfusion express a donor or third-party antigen.

In some embodiments, the method does not include host myeloablative conditioning, or includes minimal myeloablative conditioning.

In some embodiments, the priming transfusion and receptor antagonist are administered at least three days, e.g., at least four, five, six, seven or more days before the hematopoietic stem cell transplant. In some embodiments, the priming transfusion and receptor antagonist are administered concurrently or consecutively, e.g., the receptor antagonist is administered at one or more of before, concurrently with, or after the priming transfusion.

In some embodiments, the CD122 antagonist and receptor antagonist are administered before the hematopoietic stem cell transplant, e.g., at least two hours, at least one day, two days, or more, before the hematopoietic stem cell transplant. In some embodiments, the CD122 antagonist is administered in more than one dose, e.g., in at least two, three, four or more doses. In some embodiments, the CD122 antagonist and receptor antagonist are administered concurrently.

Further, the invention provides kits including a CD122 antagonist and a CD154 antagonist, and instructions for use in inducing hematopoietic chimerism and central tolerance in a transplant recipient.

Additionally, the invention provides therapeutic compositions including a CD122 antagonist and/or a CD154 antagonist, for use in a method of inducing hematopoietic chimerism and central tolerance in a transplant recipient as described herein.

A “recipient” is a subject into whom a stem cell, tissue, or organ graft is to be transplanted, is being transplanted, or has been transplanted. An “allogeneic” cell is obtained from a different individual of the same species as the recipient and expresses “alloantigens,” which differ from antigens expressed by cells of the recipient. A “xenogeneic” cell is obtained from a different species than the recipient and expresses “xenoantigens,” which differ from antigens expressed by cells of the recipient.

A “donor” is a subject from whom a stem cell, tissue, or organ graft has been, is being, or will be taken. “Donor antigens” are antigens expressed by the donor stem cells, tissue, or organ graft to be transplanted into the recipient. “Third party antigens” are antigens that differ from both antigens expressed by cells of the recipient, and antigens expressed by the donor stem cells, tissue, or organ graft to be transplanted into the recipient. The donor and/or third party antigens may be alloantigens or xenoantigens, depending upon the source of the graft. An allogeneic or xenogeneic cell administered to a recipient can express donor antigens, i.e., some or all of the same antigens present on the donor stem cells, tissue, or organ to be transplanted, or third party antigens. Allogeneic or xenogeneic cells can be obtained, e.g., from the donor of the stem cells, tissue, or organ graft, from one or more sources having common antigenic determinants with the donor, or from a third party having no or few antigenic determinants in common with the donor.

“Central tolerance” is tolerance that is established in lymphocytes developing in central lymphoid organs; “peripheral tolerance” is tolerance acquired by mature lymphocytes in the peripheral tissues.

A “hematopoietic stem cell” is a cell, e.g., a bone marrow cell, or a fetal liver or spleen cell, which is multipotent, e.g., capable of developing into multiple lineages, e.g., any myeloid and lymphoid lineages, and self-renewing, e.g., able to provide durable hematopoietic chimerism.

A compound that “specifically” binds to a target molecule is a compound that binds to the target molecule and does not substantially bind to other molecules. A “dose” of an antagonist is a therapeutically effective amount of an active compound, or a fraction thereof wherein the total amount of doses is a therapeutically effective amount. A “low dose” of bone marrow is≦about 2.5×10⁸ cells/kg.

The invention provides several advantages. First, the methods described herein require minimal or no myeloablative conditioning, which is often extremely toxic to the recipient and leaves the recipient vulnerable to infection and disease. Elimination of stringent conditioning makes central tolerance induction for facilitation of transplantation and the treatment of autoimmune disease a safer and more widely applicable clinical tool. The methods produce central tolerance that is donor-specific, while leaving the rest of the recipient's adaptive immune response intact. The central tolerance produced by the methods described herein is durable, e.g., long-lasting, and leads to long term tolerance of donated cells and tissues. This can obviate the need for life-long treatment with highly toxic immunosuppressive drugs as is typically required in conventional transplantation methods.

Furthermore, the methods generally require only a single transfusion of bone marrow, eliminating the need for repeated and painful treatments, and only a relatively low dose of bone marrow is needed, obviating the need for finding several suitably matched donors or removing bone marrow from the same donor multiple times.

In addition, the methods can be used to facilitate either allogeneic or xenogeneic transplant procedures.

Finally, unlike methods that rely on peripheral tolerance, the new methods of inducing central tolerance described herein do not rely on CD4+ cells. Persons whose transplant engraftment depends on peripheral tolerance are contingent on a viable population of CD4 cells, and are thus vulnerable to losing their graft if exposed to a CD4+ T cell-killing agent, e.g., a virus. Persons whose graft is transplanted using a method described herein and who thus have central graft tolerance would not be vulnerable to such agents, as they would have the capacity to produce new CD4+ T cells.

Unless otherwise defined, all technical and scientific terms used herein have the same meaning as commonly understood by one of ordinary skill in the art to which this invention belongs. Although methods and materials similar or equivalent to those described herein can be used in the practice or testing of the methods described herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a line graph illustrating persistence of donor-origin peripheral blood mononuclear cells (PBMC) in two groups of mice.

FIG. 2 is a line graph illustrating the deletion of peripheral host alloreactive CD8+ T cells.

FIGS. 3A and 3B are dot plots illustrating the percentage of cells expressing CD4 and CD8 in thymi recovered from untreated mice (3A) and hematopoietic chimeric mice (3B).

FIGS. 3C and 3D are histograms of the data in FIGS. 3A and 3B, respectively. The horizontal bars depict the gates used to determine the number of DES+ cells in the CD8 and CD4 quadrant.

### DETAILED DESCRIPTION

Described herein are methods for producing hematopoietic chimerism and durable central tolerance in a transplant recipient, by first inducing peripheral tolerance via costimulation blockade-based protocols, typically with minimal or no myeloablative conditioning. The induction of peripheral tolerance by treatment with a priming transfusion to activate alloreactive T cells, with administration of a blocker of a costimulatory pathway (e.g., blocking the CD40-CD154 interaction using an anti-CD154 monoclonal antibody (mAb)), facilitates stem cell engraftment and the generation of hematopoietic chimerism, leading to establishment of donor-specific central transplantation tolerance, without significant GVHD.

Costimulation blockade-based protocols have been shown to be effective for inducing peripheral transplantation tolerance (Rossini et al., 1999, supra). Previous methods use a donor-specific transfusion (DST, cells taken from the intended organ or tissue donor) to activate alloreactive T cells, with simultaneous blockade of CD40-CD154 interaction using an anti-CD154 monoclonal antibody (mAb) (Rossini et al., Physiol. Rev., 79:99-141, 1999). Such protocols have been shown to induce permanent survival of pancreatic islet allografts in mice (Parker et al., Proc. Natl. Acad. Sci. U.S.A., 92:9560-9564, 1995) and prolonged survival of skin allografts in both mice (Markees et al., Transplant. Proc., 30:2444-2446, 1998; Markees et al., J. Clin. Invest., 101:2446-2455, 1998; Markees et al., Transplantation, 64:329-335, 1997) and non-human primates (Elster et al., Transplantation, 72:1473-1478, 2001). The mechanism of peripheral transplantation tolerance induction based on DST plus anti-CD154 mAb is believed to involve the action of IFN-γ, CTLA4, regulatory CD4⁺ T cells, and the deletion of alloreactive CD8⁺ T cells (Iwakoshi et al., J. Immunol., 167:6623-6630, 2001; Iwakoshi et al., J. Immunol., 164:512-521, 2000).

As is the case in peripheral tolerance induction (Besselsen et al., Lab. Anim. Sci., 49(3):308-312, 1999), anti-CD154 mAb monotherapy in the absence of a donor-specific transfusion (DST) is generally ineffective for generating hematopoietic chimerism. Surprisingly, as demonstrated herein, third-party (e.g., MHC-disparate) transfusion (TPT) can be substituted for the standard donor-specific transfusion (DST) in “normal” recipients, i.e., recipients with a physiologically normal (low) percentage of alloreactive T cells, e.g., about 1 to 3%. Thus, the present methods include inducing hematopoietic chimerism in a normal recipient by administering a priming transfusion comprising either donor-derived cells (a donor-specific transfusion, or DST, in which the cells come from the intended donor of the hematopoietic stem cells and tissue or organ graft), or non-MHC-matched, third-party cells (a third-party transfusion, or TPT).

Synchimeric recipients, on the other hand, with supraphysiologically high levels of alloreactive T cells e.g., about 6 to 8%, became chimeric only if given a donor-specific transfusion, presumably to reduce the number of alloreactive cells. In a normal CBA/J mouse, the number of naturally occurring T cells with allospecificity is far lower, and the requirement for donor specificity of the transfusion was less stringent. Thus, the invention includes methods for determining the suitability of a recipient for the use of cells derived from a third party (a TPT) versus cells derived from the intended donor (a DST) by evaluating the recipient's levels of alloreactive T cells, and not administering the TPT to persons with high levels of alloreactive T cells. Any methods can be used to evaluate the recipient's levels of alloreactive T cells, including, but not limited to, using a mixed lymphocyte reaction (Markeed at al., J. Clin. Invest., 101:2446-2455, 1998) or an interferon-γ secretion assay (Brehm et al., J. Immunol., 170:4077-4086, 2003.

Thus, the present invention provides methods for inducing hematopoietic chimerism and central tolerance by administering to the recipient (a) a priming transfusion comprising allogeneic or xenogeneic cells, e.g., cells that express donor or third-party antigens (i.e., alloantigens) and that have a ligand on the surface that interacts with a receptor on the surface of a recipient T cell that mediates contact-dependent helper-effector function; (b) an antagonist of the receptor that inhibits interaction of the ligand with the receptor; and (c) administering a hematopoietic stem cell, e.g., bone marrow, transplant, e.g., a low-dose bone marrow transplant, thus inducing hematopoietic chimerism and central tolerance. In some embodiments, the methods further include implanting a tissue or organ graft into the recipient. The allogeneic or xenogeneic cells administered to the recipient as part of the methods described herein typically express donor antigens (i.e., some or all of the same antigens present on the donor stem cells, tissues or organ to be transplanted) or third party antigens. The allogeneic or xenogeneic cells can be obtained, e.g., from the donor of the tissue or organ graft, from one or more sources having some, all, or no common antigenic determinants with the donor, or from a third party having some, all, or no antigenic determinants in common with the donor and/or the recipient. In some embodiments, in place of or in addition to the priming transfusion of (a) above, an antagonist of CD122 is administered, e.g., an anti-CD122 antibody.

In addition to the priming transfusion, an antagonist of a molecule on T cells that mediates contact dependent helper effector functions is administered to the recipient. As defined herein, a molecule or receptor that mediates contact dependent helper effector functions is one that is expressed on a T helper (Th) cell and interacts with a ligand on an effector cell (e.g., a B cell), wherein the interaction of the molecule with its ligand is necessary for generation of an effector cell response (e.g., B cell activation). In addition to being involved in effector cell responses, it has now been found that such a molecule or receptor is involved in the response of the T cell to antigens. Typically, the molecule on a T cell that mediates contact-dependent helper effector function is CD40. Accordingly, in some embodiments, the methods described herein involve administering to a transplant recipient an allogeneic or xenogeneic cell and a CD40 antagonist. Activation of recipient T cells by the allogeneic or xenogeneic cell involves an interaction between CD40 on recipient T cells and a CD40 ligand (CD40L) on the allogeneic or xenogeneic cell. By inhibiting this interaction with a CD40 antagonist, the T cells of the recipient are not activated by the donor/third party antigens expressed by the allogeneic or xenogeneic cell, but rather become tolerized to the donor/third-party antigens. Induction of peripheral tolerance to donor antigens in the recipient thus enables successful transplantation of bone marrow without immune-mediated rejection of the donor cells. Subsequently, the recipient develops hematopoietic chimerism and central tolerance, which allows for transplantation of other tissues or organs without immune-mediated rejection.

The methods described herein further include the administration of a hematopoietic stem cell transplant, e.g., a bone marrow graft.

In some embodiments, the methods described herein include the use of minimal myeloablative conditioning of the recipient. In some embodiments, minimal myeloablative conditioning can include the use, e.g., transitory use, of low doses of one or more chemotherapy agents, e.g., vincristine, actinomycin D, chlorambucil, vinblastine, procarbazine, prednisolone, cyclophosphamide, doxorubicin, vincristine, prednisolone, lomustine, and/or irradiating the thymus of the recipient mammal, e.g., human, with a low dose of radiation, e.g., less than a lethal dose of radiation plus chemotherapy agents. Lethal doses of conditioning include the administration of 14 Gy of irradiation plus cytarabine, cyclophosphamide, and methylprednisolone (Guinin et al, New Engl. J. Med., 340:1704-1714, 1999).

To prevent the development of graft-versus-host disease, additional treatment with a short course of methotrexate and cyclosporine starting on the day before transplantation using a bolus of 1.5 mg/kg over a period of 2-3 hours every 12 hours. This protocol should allow the reduction of irradiation conditioning to about 10 Gy or less, e.g., in some embodiments, about 5 Gy, about 2 Gy, about 1.5 Gy, about 1 Gy, about 0.5 Gy, about 0.25 Gy and the elimination of additional cytoreduction agents such as cytarabine, cyclophosphamide, and methylprednisolone treatments. Minimal myeloablative conditioning is typically achieved by administering chemical or radiation therapy at a level that will not destroy the recipient's immune function, and is similar to, or lower than, levels used for conventional cancer treatments, e.g., conventional chemotherapy.

When combined with minimal myeloablative conditioning (e.g., 1 Gy/mouse), chimerism was achieved in 100% of treated recipients. As described herein, using minimal conditioning led to only a modest increase in the number of mice that were successfully engrafted, and, in those mice that were chimeric, to only a modest increase in the percentage of donor-origin PBMCs. Those effects could be mimicked in large measure by increasing the dose of bone marrow cells. Hematopoietic chimerism in mice treated with DST and anti-CD154 mAb, but no conditioning was stable over time, and under all conditions chimeric recipients appeared free of graft vs. host disease (GVHD). Thus, typically, the present methods are performed without myeloablative host conditioning.

As one theory, not meant to be limiting, it is believed that the underlying mechanism by which the methods described herein induce hematopoietic chimerism and central tolerance involves deletion of host alloreactive cells in both the thymus and the periphery of chimeric recipients; DES⁺CD8⁺CD4⁻ alloreactive T cells in the thymus of KB5 synchimeras that were chimeric for C57BL/6 hematopoietic cells are deleted.

The long-term durable hematopoietic chimerism described herein is evidence of a state of donor-specific central tolerance. Consistent with this inference, donor-specific skin allograft survival in chimeric mice was also observed.

To be of value in clinical medicine, transplantation tolerance induction procedures should be generally applicable to a broad range of recipients. In animal models, peripheral costimulation blockade-based protocols work to varying degrees depending on the host strain (Williams et al., J. Immunol., 165:6849-6857, 2002). In contrast, the methods described herein established hematopoietic cell engraftment in the absence of host conditioning in a number of different mouse strains, each of which was fully MHC-mismatched with its bone marrow donor (see Examples, below). All of the strains tested also exhibited prolonged skin allograft survival after treatment with DST plus anti-CD154 mAb.

In addition, the present methods typically include the use of lower and fewer doses of costimulation blocking agent, e.g., anti-CD154 monoclonal antibody (mAb), than previously described. For example, the total dose of anti-CD154 mAb (4 mg) in the protocol described in Durham et al., J. Immunol., 165:1-4 (2000) is 4 times larger than that used in the present experiments, and was given over 3 months rather than 2 weeks. The modest dose of anti-CD154 mAb used in the present methods is advantageous, as in human studies, anti-human CD154 mAb administered chronically over long periods of time has been associated with the development of both arterial and venous thrombosis (Buhler et al., Transplantation, 71:491, 2001; Kawai et al., Nat. Med., 6:114, 2000), possibly related to the fact that CD154 is expressed on activated platelets and may stabilize thrombi (Henn et al., Nature, 391:591-594, 1998; Andre et al., Nature Med., 8:247-252, 2002). The methods described herein can include, for example, a brief two week course of treatment with this costimulation blocking reagent to achieve a maximum beneficial effect with respect to the generation of chimerism and may avoid this potential therapeutic complication. Thus, the present methods include the administration of anti-CD154 mAb for a period of about two weeks or less.

An issue relevant to the use of multi-stage transplantation tolerance induction procedures in clinical medicine is the stringency with which the components of the therapy need to be timed. As described herein, the present methods are more successful if initiated 1 to 2 weeks before bone marrow transplantation, but less successful if initiated five, three or fewer days before transplantation. Thus, the present methods include administration of a priming transfusion at least three, four, five, six, seven, eight, nine, ten, eleven, twelve, thirteen, fourteen or more days prior to bone marrow transplantation.

Replacement of the priming transfusion (e.g., DST) with depleting anti-CD8 mAb in combination with anti-CD154 mAb prolongs skin allograft survival (Iwakoshi et al., J. Immunol., 164:512-521, 2000). However, as described herein, this strategy significantly degraded the clinical outcome when applied to bone marrow transplantation. Given that CD8⁺ T cell depletion appeared not to be the entire story, the potential role of NK cells, which have a major role in the rejection of allogeneic bone marrow cells in lethally irradiated mice, was investigated (Yu et al., Ann. Rev. Immunol., 10:189-213, 1992; Murphy et al., J. Natl. Cancer Inst., 85:1475-1482, 1993; Murphy et al., J. Exp. Med., 165:1212-1217, 1987; and Cudkowicz and Bennett, J. Exp. Med., 134:83-102, 1971).

As described herein, NK cells are important regulators of bone marrow cell engraftment in non-myeloablated mice treated with costimulation blockade. The anti-CD122 mAb is directed against the IL-2 receptor beta chain expressed on almost all NK cells and on a subpopulation of CD8⁺ T cells and activated macrophages (Tanaka et al., J. Immunol., 147:2222-2228, 1991; Ohashi et al., J. Immunol., 143:3548-3555, 1989; Allouche et al., Leuk. Res., 14:699-703, 1990). As one theory, not meant to be limiting, because CD154 is also expressed on NK cells, engraftment of allogeneic bone marrow cells in non-myeloablated hosts may require not only the deletion of donor-reactive CD8⁺ T cells, but also inactivation of host donor-reactive NK cells. Alternatively, failure of anti-CD154 mAb plus anti-CD8 mAb therapy to induce high levels of chimerism may be due to the ability of anti-CD8 mAb to delete not only the endogenous population of CD8⁺ cells, but also the exogenous population of CD8+ “facilitator” cells present in the donor bone marrow, that can enhance allogeneic hematopoietic stem cell engraftment (Schuchert et al., Nat. Med., 6:904-909, 2000; Kaufman et al., Blood, 84:2436-2446, 1994; and Fowler et al., Blood, 91:4045-4050, 1998). Anti-CD8 mAb in the host may be deleting donor facilitator cells required for stem cell engraftment in a non-myeloablated host. As described herein, mice treated with anti-CD154 mAb combined with anti-CD122 monoclonal antibody (mAb) could readily be engrafted with allogeneic bone marrow. Thus, in some embodiments, the methods include administering to the recipient a CD122 antagonist, e.g., an anti-CD122 monoclonal antibody, in addition to or in place of a priming transfusion. The methods can also include the use of any treatment that achieves the same result as the administration of an anti-CD122 antibody or the priming transfusion. The CD122 antagonist can be administered up to the time of the transplant. In some embodiments, a CD122 antagonist is administered concurrently with a CD154 antagonist, and a stem cell transplant (and, in some embodiments, a tissue or organ graft) is administered within a few (e.g., at least two, e.g., three four or more) hours or days thereafter.

The results described herein regarding the role of CD8⁺ cells in transplantation tolerance indicate that the mechanisms responsible for peripheral tolerance induction and the generation of hematopoietic chimerism and central tolerance are overlapping but different. Another distinction between the two relates to the CD4⁺ cell populations. Treatment with anti-CD4 mAb prevents the induction of peripheral transplantation tolerance by DST plus anti-CD154 mAb (Markees et al., J. Clin. Invest., 101:2446-2455, 1998; and Iwakoshi et al., J. Immunol., 167:6623-6630, 2001). In contrast, the addition of anti-CD4 mAb to the chimerism protocol described herein did not prevent hematopoietic engraftment, although it did reduce the level of chimerism.

The methods described herein permit allogeneic bone marrow cell engraftment and the generation of hematopoietic chimerism. The results described herein suggest that there is commonality to the generation of peripheral and central tolerance, and that the maintenance of transplantation tolerance will require either physical thymectomy or its biological equivalent-central tolerance induction. The methods described herein can be practiced without host myeloablative conditioning, appear not to cause GVHD, and in some cases do not even require MHC matching of priming transfusion, bone marrow, and donor organs; these characteristics make the methods described herein highly useful in clinical medicine.

Various aspects of the invention are described in further detail in the following subsections.

I. CD154 and CD122 Antagonists

The methods described herein include the administration of antagonists to CD154, and, in some embodiments, CD122.

A. Blockers of Costimulation: CD154 Antagonists

CD154 is a 39 kDa transmembrane glycoprotein also known as gp39 and CD40 ligand (or CD40L).

According to the methods described herein, a CD154 antagonist is administered to a recipient to interfere with the interaction of CD154 on recipient T cells with a CD154 ligand (e.g., CD40) on an allogeneic or xenogeneic cell, such as a B cell, administered to the recipient. A CD154 antagonist is defined as a molecule that interferes with this interaction. The CD154 antagonist can be, e.g., an antibody directed against CD154 (e.g., a monoclonal antibody against CD154), a fragment or derivative of an antibody directed against CD154 (e.g., Fab or F(ab′)₂ fragments, chimeric antibodies or humanized antibodies), soluble forms of a CD154 ligand (e.g., soluble CD40), soluble forms of a fusion protein of a CD154 ligand (e.g., soluble CD40Ig), or pharmaceutical agents that disrupt or interfere with the CD154-CD40 interaction. Alternatively, the CD154 antagonist can be an anti-CD40 antibody.

B. Recipient-specific CD8+ Cell Deletion: CD122 Antagonists

CD122, also known as interleukin 2 receptor beta chain or IL-2 Rbeta, is one of the critical subunits of IL-2R and IL-15R, and is crucial in IL-2 and IL-15-mediated signaling. CD122 is a 70-75 kDa protein, long single chain type I transmembrane molecule of about 525 amino acids. See Minami et al., Annu. Rev. Immunol., 11:245-68, 1993. As one theory, not meant to be limiting, administration of a CD122 antagonist to a subject selectively depletes donor-reactive CD8+ and NK cells that originated in the recipient as opposed to the donor, while leaving substantially intact those CD8+ donor facilitator cells required for stem cell engraftment in a non-myeloablated host. In some embodiments, the CD122 antagonist is an anti-CD122 antibody or antigen-binding portion thereof.

C. Anti-CD122, -CD40, and -CD154 Antibodies

In some embodiments, an antagonist (e.g., a CD154 or CD122 antagonist) can be an antibody, e.g., an antibody against CD154 or CD40, or against CD122. The term “antibody” as used herein includes polyclonal, monoclonal, monospecific, chimeric, humanized, de-immunized, or other modified antibodies, and antigen-binding fragments thereof, that specifically bind to a CD122, CD154 or CD40 protein or peptide thereof, or a CD122, CD154 or CD40 fusion protein.

Antibodies can be fragmented using conventional techniques and the fragments screened for utility using methods known in the art, e.g., as described herein for whole antibodies. For example, F(ab′)₂ fragments can be generated by treating an antibody with pepsin. The resulting F(ab′)₂ fragment can be treated to reduce disulfide bridges to produce Fab′ fragments. The antibodies described herein can include bispecific and chimeric molecules having an anti-CD122, anti-CD154, or anti-CD40 portion.

When antibodies produced in non-human subjects are used therapeutically in humans, they are often recognized to varying degrees as foreign and an immune response may be generated in the patient. One approach for minimizing or eliminating this problem, which is preferable to general immunosuppression, is to produce chimeric antibody derivatives, i.e., antibody molecules that combine a non-human animal variable region and a human constant region. Chimeric antibody molecules can include, for example, the antigen binding domain from an antibody of a mouse, rat, or other species, with human constant regions. A variety of approaches for making chimeric antibodies have been described and can be used to make chimeric antibodies containing the immunoglobulin variable region that recognizes CD122, CD154 or CD40. See, for example, Morrison et al., Proc. Natl. Acad. Sci. U.S.A., 81:6851 (1985); Takeda et al., Nature, 314:452 (1985), Cabilly et al., U.S. Pat. No. 4,816,567; Boss et al., U.S. Pat. No. 4,816,397; Tanaguchi et al., European Patent Publication EP 171496; Morrison et al., European Patent Publication 0173494; and Rabbitts et al., United Kingdom Patent No. GB 2177096B. It is expected that such chimeric antibodies would be less immunogenic in a human subject than the corresponding non-chimeric antibody.

For human therapeutic purposes, monoclonal or chimeric antibodies specifically that specifically bind to a CD122, CD154, or CD40 protein or peptide can be further humanized by producing human variable region chimeras, in which parts of the variable regions, especially the conserved framework regions of the antigen-binding domain, are of human origin and only the hypervariable regions are of non-human origin. Such altered immunoglobulin molecules may be made by any of several techniques known in the art, (e.g., Teng et al., Proc. Natl. Acad. Sci. U.S.A., 80:7308-7312 (1983); Kozbor et al., Immunology Today, 4:7279 (1983); and Olsson et al., Meth. Enzymol., 92:3-16 (1982)), and can also be made according to the methods of PCT Publication W092/06193 or EP 0239400. Humanized antibodies can be commercially produced by, for example, Scotgen Limited, 2 Holly Road, Twickenham, Middlesex, Great Britain.

Another method of generating specific antibodies or antibody fragments that specifically bind to a CD122, CD154, or CD40 protein or peptide is to screen expression libraries encoding immunoglobulin genes, or portions thereof, expressed in bacteria with a CD122, CD154 or CD40 protein or peptide. For example, complete Fab fragments, VH regions and FV regions can be expressed in bacteria using phage expression libraries. See for example Ward et al., Nature, 341: 544-546: (1989); Huse et al., Science, 246: 1275-1281 (1989); and McCafferty et al., Nature, 348: 552-554 (1990). Screening such libraries with, for example, a CD122, CD154, or CD40 peptide can identify immunoglobin fragments that specifically bind to CD122, CD154 or CD40, respectively. Alternatively, the SCID-hu mouse (a severe combined immunodeficient (SCID) mouse transplanted with human fetal thymus and liver tissues, available from Genpharm, San Jose, Calif., or Advanced Bioscience Resource, Alameda, Calif.; See Brooks et al., Nat. Med., 7:459-464, 2001) can be used to produce antibodies, or fragments thereof.

A number of anti-CD154 antibodies are known in the art, or can be generated using known methods, e.g., as described in U.S. Pat. No. 5,902,585 to Noelle et al.

Methodologies for producing monoclonal antibodies directed against CD154, including human CD154 and mouse CD154, and suitable monoclonal antibodies for use in the new methods, are described below and in detail in Example 2 of U.S. Pat. No. 5,902,585.

In some embodiments, the anti-CD154 antibody is BG9588 (hu5C8, Biogen, Cambridge, Mass.), a recombinant humanized monoclonal antibody, or IDEC-131 (IDEC Pharmaceuticals, San Diego, Calif.).

In some embodiments, the CD154 antagonist can be an anti-CD40 antibody (e.g., as described in Pearson et al., Transplantation, 74:933-940, 2002; Haanstra et al., Transplantation, 75:637-643, 2003).

In some embodiments, the CD122 antagonist is an anti-CD122 antibody, e.g., Mik-beta 1 (Tsudo et al., Proc Natl Acad Sci U S A., 86(6):1982-6, 1989) or TU27 (Takeshita et al., J Exp Med., 169(4):1323-32, 1989).

D. Soluble Ligands for CD154

Other CD154 antagonists that can be administered to induce T cell tolerance include soluble forms of a CD154 ligand. A monovalent soluble ligand of CD154, such as soluble CD40, can bind to CD154, thereby inhibiting the interaction of CD154 with CD40 on B cells. The term “soluble” indicates that the ligand is not permanently associated with a cell membrane. A soluble CD154 ligand can be prepared by chemical synthesis, or by recombinant DNA techniques, for example by expressing only the extracellular domain (absent the transmembrane and cytoplasmic domains) of a ligand. One example of a soluble CD154 ligand is soluble CD40. Alternatively, a soluble CD154 ligand can be in the form of a fusion protein. Such a fusion protein typically comprises at least a portion of the CD154 ligand attached to a second molecule. For example, CD40 can be expressed as a fusion protein with immunoglobulin (i.e., a CD40Ig fusion protein). In one embodiment, a fusion protein is produced comprising amino acid residues of an extracellular domain portion of CD40 joined to amino acid residues of a sequence corresponding to the hinge, CH2 and CH3 regions of an immunoglobulin heavy chain, e.g., Cyl, to form a CD40Ig fusion protein (see e.g., Linsley et al., J. Exp. Med., 1783:721-730, 1991; Capon et al., Nature, 337, 525-531, 1989; and Capon U.S. Pat. No. 5,116,964). The fusion protein can be produced by chemical synthesis, or by recombinant DNA techniques, e.g., based on the cDNA of CD40 (Stamenkovic et al., EMBO J., 8:1403-1410,1989.

II. Cells for Use in Priming Transfusion

The present methods can include the administration of a priming transfusion of allogeneic or xenogeneic cells. As one theory, the presentation of alloantigens to recipient T cells in the presence of a CD154 antagonist induces peripheral T cell tolerance to the alloantigens. Cells that are capable of inducing tolerance by this mechanism include those that present antigen and activate T cells by interaction with CD154 (i.e., an interaction between CD154 on T cells and a CD154 ligand on the antigen-presenting cell is necessary to deliver the appropriate signals for T cell activation to the T cell). Inhibition of the interaction of the ligand on the allogeneic or xenogeneic cell with CD154 on recipient T cells prevents T cell activation by allo- or xenoantigens and, rather, induces T cell tolerance to the antigens. As one theory, not meant to be limiting, interference with activation of the T cell via CD154 may prevent the induction of costimulatory molecules on the allogeneic or xenogeneic cell, (e.g., B7 family molecules on a B cell), so that the cell delivers only an antigenic signal to the T cell in the absence of a costimulatory signal, thus inducing tolerance.

Accordingly, an allogeneic or xenogeneic cell is administered to a recipient subject. The allogeneic or xenogeneic cell is capable of presenting an antigen to T cells of the recipient, and is, for example, a B lymphocyte, a “professional” antigen presenting cell (e.g., a monocyte, dendritic cell, or Langerhans cell) or other cell that can present antigen to immune cells (e.g., a keratinocyte, endothelial cell, astrocyte, fibroblast, or oligodendrocyte). Typically, the allogeneic or xenogeneic cell has a reduced capacity to stimulate a costimulatory signal in recipient T cells. For example, the allogeneic or xenogeneic cell can lack expression of, or express only low levels of, costimulatory molecules such as the B7 family of proteins (e.g., B7-1 and B7-2), e.g., naturally or as a result of genetic engineering using methods known in the art (e.g., temporary methods such as antisense or RNAi, or by stable expression of a B7-1 or B7-2 knockout). Expression of costimulatory molecules on potential allogeneic or xenogeneic cells to be used in the methods described herein can be assessed by standard techniques, for example by flow cytometry using antibodies directed against the costimulatory molecules.

Allogeneic or xenogeneic cells suitable for inducing T cell tolerance include lymphoid cells, for example peripheral blood lymphocytes or splenic cells, e.g., B cells. B cells can be purified from a mixed population of cells (e.g., other cell types in peripheral blood or spleen) by standard cell separation techniques. For example, adherent cells can be removed by culturing spleen cells on plastic dishes and recovering the non-adherent cell population. T cells can be removed from a mixed population of cells by treatment with an anti-T cell antibody (e.g., anti-Thy1.1 and/or anti-Thy1.2) and complement.

In one embodiment, resting lymphoid cells, e.g., resting B cells, are used as the antigen presenting cells. Resting lymphoid cells, such as resting B cells, can be isolated by techniques known in the art, for example based upon their small size and density. Resting lymphoid cells can be isolated for example by counterflow centrifugal elutriation, e.g., as known in the art and/or as described in Example 1 of U.S. Pat. No. 5,902,585. Using counterflow centrifugal elutriation, a small, resting lymphoid cell population depleted of cells that can activate T cell responses can be obtained by collecting a fraction(s) at 14-19 ml/min., e.g., 19 ml/min. (at 3,200 rpm). Alternatively, small, resting lymphocytes (e.g., B cells) can be isolated by discontinuous density gradient centrifugation, for example by using a Ficoll or Percoll gradient, a layer containing small, resting lymphocytes can be obtained after centrifugation. Small resting B cells can also be distinguished from activated B cells by assaying for expression of costimulatory molecules, such as B7-1 and/or B7-2, on the surface of activated B cells by standard techniques (e.g., immunofluorescence).

The allogeneic or xenogeneic cells administered to the recipient function, at least in part, to present donor and/or third-party antigens to recipient T cells. Thus, in some embodiments, the cells express antigens that are also expressed by the donor tissue or organ. Typically, this can be accomplished by using allogeneic or xenogeneic cells obtained from the donor of the stem cells and/or tissue or organ graft. For example, peripheral lymphoid cells, B cells, or spleen cells from the stem cell, tissue, or organ donor can be isolated and used in the methods described herein. Alternatively, allogeneic or xenogeneic cells can be obtained from a source other than the donor of the bone marrow, tissue, or organ, e.g., a third party. In some embodiments, the cells have antigenic determinants in common with the bone marrow, tissue, or organ donor. For example, allogeneic or xenogeneic cells that express (most or all) of the same major histocompatibility complex antigens as the donor tissue or organ can be used. Thus, allogeneic or xenogeneic cells may be used from a source that is MHC haplotype matched with the donor of the bone marrow, tissue or organ (e.g., a close relative of the graft donor). In other embodiments, the cells have antigenic determinants that differ from one or more of the bone marrow and/or tissue or organ donor, and the recipient. Thus, allogeneic or xenogeneic cells may be used from a source that is MHC haplotype mismatched with one or more of the donor of the bone marrow, tissue or organ, and the recipient.

Typically, the donor of the bone marrow will also be the donor of any subsequent issue or organ graft, e.g., where the donor is a living, viable human being, e.g., a voluntary organ donor.

The surprising discovery that the priming transfusion given after the first injection of anti-CD154 mAb need not be MHC-matched with the eventual bone marrow donor (i.e., not “donor specific”) has important clinical and theoretical implications. First, the complex orchestration of obtaining and delivering to a recipient a priming transfusion and bone marrow and an organ for transplantation can now be greatly simplified. Mechanistically, the kinetics of susceptibility to engraftment may relate to the ability of a non-allo-matched third party transfusion (TPT) to induce “non-specific” regulatory mechanism(s) that facilitate the process.

There are a number of theories regarding the effects of donor-lymphocyte transfusion as a means of enhancing allograft survival. Proposed mechanisms include 1) establishment of mixed allogeneic chimerism (Sykes, Immunity, 14:417-424, 2001; and De Waal and van Twuyver, Crit. Rev. Immunol., 10:417-425, 1991) deletion of donor-reactive T cells (Iwakoshi et al., J. Immunol., 164:512-521, 2000; Iwakoshi et al., J. Immunol., 167:6623-6630, 2001; Li et al., Nat. Med., 5:1298-1302, 1999; Wells et al., Nat. Med., 5:1303-1307, 1999; and Trambley et al., J. Clin. Invest., 104:1715-1722, 1999), induction of clonal anergy (Dallman et al., J. Exp. Med., 173:79-87, 1991), cytokine production (Josien et al., Transplantation, 60:1131-1139, 1995), and the generation of regulatory T cells (Markees et al., J. Clin. Invest., 101:2446-2455, 1998; Iwakoshi et al., J. Immunol., 167:6623-6630, 2001; Yang et al., Blood, 91:324-330, 1998; and Vignes et al., J. Immunol., 165:96-101, 2000).

An additional possibility is the induction of tolerogenic dendritic cells (Homann et al., Immunity, 16:403-415, 2002; Grohmann et al., J. Immunol., 166:277-283, 2001; Hawiger et al., J. Exp. Med., 194:769-779, 2001; and Miga et al., Eur. J. Immunol., 31:959-965, 2001) and/or the production of regulatory cytokines by an immune system activated in the presence of anti-CD154 mAb. Dendritic cells that ingest apoptotic cells (Stuart et al., J. Immunol., 168:1627-1635, 2002) (as the priming transfusion is eliminated) or become activated in the presence of CD40-CD154 blockade appear to become tolerogenic cells that suppress immune responses and secrete regulatory cytokines such as TGF-β and IL-10 (Hara et al., J. Immunol.,166:3789-3796, 2001; and Zeller et al., J. Immunol., 163:3684-3691, 1999). Alternatively, the requirement for the at least about 7 day delay after DST for bone marrow engraftment to occur may possibly be due to delayed deletion of host alloreactive T cells. It is known that fully-activated CD8⁺ T cells migrate to non-lymphoid tissues where they become memory cells (Lefrancois and Masopust, Curr. Opin. Immunol., 14:503-508, 2002; Kim et al., J. Immunol., 159:4295-4306, 1997). Incomplete activation in the presence of CD40-CD154 blockade may induce migration and initiate apoptosis of antigen-activated T cells that could be reversed if a second allo-stimulus in the form of allogeneic bone marrow is given too soon after the priming transfusion.

III. Administration of Priming Transfusion and/or Antagonists

Durable central tolerance to an organ or tissue graft can be induced according to the methods described herein by administration to the transplant recipient of a CD154 antagonist in conjunction with (i) a priming transfusion of allogeneic or xenogeneic cells that express donor and/or third-party antigens and interact with recipient T cells via CD154 and/or (ii) a CD122 antagonist, e.g., an anti-CD122 antibody, followed by transplantation of hematopoietic stem cells, e.g., bone marrow.

In some embodiments, the CD154 antagonist, and the priming transfusion and/or CD122 antagonist, are administered to the recipient essentially simultaneously or contemporaneously. Alternatively, the CD154 antagonist can be administered prior to administering the allogeneic or xenogeneic cells and/or CD122 antagonist, for example when the CD154 antagonist is an antibody with a long half-life.

In some embodiments, the CD154 antagonist is administered in multiple doses, e.g., two, three, four, or more doses, e.g., before, concurrently with, and/or after the administration of the priming transfusion. As one example, not meant to be limiting, one dose can be administered with the priming transfusion, and additional doses can be administered, e.g., about every day or every two, three, or four days after that.

In some embodiments, the CD122 antagonist is administered in multiple doses, e.g., two, three, four, or more doses, e.g., before, concurrently with, and/or after the administration of the priming transfusion. As one example, not meant to be limiting, one dose can be administered with the priming transfusion, and additional doses can be administered, e.g., about every day or every two, three, or four days after that. In some embodiments, the CD122 antagonist is administered at about seven days, or is administered in two doses, e.g., one dose at about seven days and a second dose at about one day. In some embodiments, the CD122 antagonist is administered at least once, about a few (e.g., at least two, e.g., three, four, or more) hours before the bone marrow transplant. In some embodiments, the CD122 antagonist is administered in two doses, at about seven days and about one day before the bone marrow transplant.

In some embodiments, the allogeneic or xenogeneic cells and/or dose or doses of CD122 antagonist, and the dose or doses of CD154 antagonist, are administered to the recipient prior to transplantation of the stem cells, e.g., bone marrow, into the recipient (i.e., the recipient is pretreated with the cells and the antagonist). For example, administration of the allogeneic or xenogeneic cells can be performed several days (e.g., at least seven days, e.g., about seven, eight, nine, ten, eleven, twelve, thirteen, fourteen or more days) prior to stem cell transplantation.

In some embodiments, the methods include the administration of an additional one or more doses of the CD154 and/or CD122 antagonist concurrently with, and/or subsequent to, the administration of the bone marrow transplant.

Administration of a single dose of allogeneic cells (e.g., in combination with a CD154 antagonist) has been found to be sufficient for use in the present methods. The number of cells administered can vary depending upon the type of cell used, the type of tissue or organ graft, the weight of the recipient, the general condition of the recipient and other variables known to the skilled artisan. An appropriate number of cells for use in the methods described herein can be determined by one of ordinary skill in the art by conventional methods (for example as described in Example 1 of U.S. Pat. No. 5,902,585). For example, where the recipient is human, about 1.0×10⁵ to about 1.0×10⁹ cells can be administered. Cells are typically administered in a form and by a route that is suitable for induction of T cell tolerance in the recipient, e.g., intravenously. Cells can be administered in a physiologically acceptable solution, such as a buffered saline solution or similar vehicle.

An antagonist, e.g., a CD122 or CD154 antagonist, as described herein is typically administered to a subject in a biologically compatible form suitable for pharmaceutical administration in vivo to induce T cell tolerance, e.g., in a therapeutic composition. A “biologically compatible form suitable for administration in vivo” is a form of the antagonist to be administered in which any toxic effects are outweighed by the therapeutic effects of the compound. The term “subject” includes living organisms in which an immune response can be elicited, e.g., mammals. Examples of subjects include humans, dogs, cats, horses, rabbits, cows, sheep, goats, pigs, mice, rats, and transgenic species thereof. An antagonist as described herein can be administered in any pharmacologically acceptable form, optionally in a pharmaceutically acceptable carrier. As used herein “pharmaceutically acceptable carrier” includes any and all solvents, dispersion media, coatings, antibacterial and antifungal agents, isotonic and absorption delaying agents. and the like. The use of such media and agents for pharmaceutically active substances is well known in the art.

Administration of a therapeutically active amount of an antagonist is defined as an amount effective, at dosages and for periods of time necessary to achieve the desired result (e.g., tolerance). In some embodiments, a therapeutically active amount is about 0.05 mg/kg, about 0.25 mg/kg, about 1.0 mg/kg, about 5.0, about 10 mg/kg, about 15 mg/kg, about 20 mg/kg, about 25 mg/kg, or about 30 mg/kg or more. For example, a therapeutically active amount of an antagonist can vary according to factors such as the nature of the antagonist, the disease state, age, sex, and weight of the individual, and the ability of the antagonist to elicit a desired response in the individual. Dosage regimens may be adjusted to provide the optimum therapeutic response. For example, several divided doses can be administered daily or the dose can be proportionally reduced as indicated by the exigencies of the therapeutic situation. An effective treatment regimen can include initiation of antibody administration prior to tissue or organ transplantation (e.g., seven or more days before transplantation), followed by re-administration of the antibody (e.g., at regular intervals of every other day, every two, three, or four days, etc., or irregular intervals) for several weeks (e.g., two to seven weeks) after transplantation. In some embodiments, the dosage is administered as a single intravenous infusion. In some embodiments, the dosage is about 10-30 mg/kg administered by IV infusion once every 14 days for about two to three doses, and then once about every 14 to 28 days for about two, three, or four more doses.

The antagonist, e.g., an antibody or antigen-binding fragment thereof can be administered in any convenient manner, e.g., by injection (subcutaneous, intravenous, etc.), oral administration, inhalation, transdermal application, or rectal administration. Depending on the route of administration, the active compound can be coated in a material to protect the compound from the action of enzymes, acids and other natural conditions that may inactivate the compound. Typically, the route of administration will be by intravenous injection.

To administer an antagonist by other than parenteral administration, it may be necessary to coat the antagonist with, or co-administer the antagonist with, a material to prevent its inactivation. For example, an antagonist can be administered to an individual in an appropriate carrier or diluent, co-administered with enzyme inhibitors or in an appropriate carrier such as liposomes. Pharmaceutically acceptable diluents include saline and aqueous buffer solutions. Enzyme inhibitors include pancreatic trypsin inhibitor, diisopropylfluorophosphate (DEP) and trasylol. Liposomes include water-in-oil-in-water emulsions as well as conventional liposomes (Strejan et al., J. Neuroimmunol., 7:27, 1984).

The active compound can also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. Typically, the composition will be sterile and will be sufficiently fluid to allow for easy syringability. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols such as mannitol, sorbitol, sodium chloride will be included in the composition. Prolonged absorption of the injectable compositions can be brought about by including in the composition an agent that delays absorption, for example, aluminum monostearate and gelatin. Except insofar as any conventional media or agent is incompatible with the active compound, use thereof in the therapeutic compositions is contemplated. Supplementary active compounds can also be incorporated into the compositions, e.g., second therapeutic agents, e.g., antibiotics, or other immunosuppressive drugs, e.g., rapamycin, mycophenolate, mofetil, anti-thymocyte sera, anti-CD45RB antibody, and/or anti-LFA antibody.

Sterile injectable solutions can be prepared by methods known in the art, e.g., by incorporating an antagonist in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, typical methods of preparation are vacuum drying and freeze-drying which yields a powder of the antagonist plus any additional desired ingredient from a previously sterile-filtered solution thereof.

In some embodiments, parenteral compositions can be formulated in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

A suitably prepared protein can also be mucosally administered, for example, orally administered with an inert diluent or an assimilable edible carrier.

Subsequent to or concurrent with the methods for the induction of central tolerance described herein, a donor tissue or organ can be transplanted into a transplant recipient by conventional techniques.

IV. Hematopoietic Stem Cell Transplant

The present methods include the administration of a hematopoietic stem cell graft to the recipient. In some embodiments, the stem cells are, or are derived from, bone marrow. As noted above, hematopoietic stem cells are cells, e.g., bone marrow cells, or fetal liver or spleen cells, which are multipotent, e.g., capable of developing into multiple or all myeloid and lymphoid lineages, and self-renewing, e.g., able to provide durable hematopoietic chimerism. Purified preparations of hematopoietic cells or mixed preparations, such as bone marrow, which include other cell types, can be used in the methods described herein. The preparation typically includes immature cells, i.e., undifferentiated hematopoietic stem cells; a substantially pure preparation of stem cells can be administered, or a complex preparation including other cell types can be administered. As one example, in the case of bone marrow stem cells, the stem cells can be separated out to form a pure preparation, or a complex bone marrow sample including stem cells can be used as a mixed preparation. Hematopoietic stem cells can be from fetal, neonatal, immature, or mature animals. Typically, peripheral blood hematopoietic stem cells derived from the intended tissue or organ donor will be used. Methods for the preparation and administration of hematopoietic stem cell transplants are known in the art, e.g., as described in U.S. Pat. Nos. 6,514,513 and 6, 208,957. For example, stem cells can be derived from peripheral blood (Burt et al., Blood, 92:3505-3514, 1998), cord blood (Broxmeyer et al., Proc. Nat. Acad. Sci. U.S.A., 86:3828-3832, 1989), bone marrow (Bensinger et al., New Eng. J. Med., 344:175-181, 2001), and/or and embryonic stem cells (Palacios et al., Proc. Nat. Acad. Sci. U.S.A., 92:7530-7534, 1995).

In some embodiments, the methods described herein include the use of a single dose of bone marrow. In the animal models described herein, an allogeneic bone marrow dose of 50×10⁶ cells per recipient mouse, in the absence of myeloablative conditioning, efficiently generated robust chimerism. Minimal preparative myeloablative conditioning with 1 Gy of radiation and 50% fewer bone marrow cells (25×10⁶ cells/mouse) also generated robust chimerism, with recipients uniformly circulating approximately 20% donor-origin PBMC. Any suitable method can be used for minimal conditioning, e.g., minimal myeloablation using low dose irradiation or chemotherapeutic agents, e.g., as described herein. One of skill in the art will appreciate that the size of the stem cell inoculum can thus be balanced with the intensity of preparative conditioning (if any). Previously described methods used either (1) much larger doses or (2) multiple doses of bone marrow. One protocol used a single injection of 200×10⁶ bone marrow cells/mouse (four times larger than the dose used herein) (Wekerle et al., Nat. Med., 6:464-469, 2000). Another protocol used 8 injections of 2×10⁷ bone marrow cells plus anti-CD154 mAb (Durham et al., J. Immunol., 165:1-4, 2000); the total number of bone marrow cells (160×10⁶) was ˜3 times larger than that used in the experiments described herein.

A living human donor can provide about 7.5×10⁸ bone marrow cells/kg. The methods described herein can include the administration of at least 2 or 3 times this number (per kg) or more, e.g., 2.5×10⁸ cells/kg (i.e.,a “low dose”) up to about 6×10⁸ cells/kg (Rocha et al., J. Clin. Onc., 20:4324-4330, 2002; Dominietto et al., Blood, 100:3930-3934, 2002; and Li et al., J. Pediatr. Child Health, 38:308-310, 2002). The requisite numbers of bone marrow cells can be provided by the ex vivo expansion or amplification of human stem cells, e.g., as reviewed in Emerson, Blood, 87(8):3082-8, 1996, and described in more detail in Petzer et al., Proc. Natl. Acad. Sci. U.S.A., 93(4):1470-4, 1996; Zandstra et al., BioTechnology, 12(9):909-14, 1994; and Davis et al., PCT Publication, WO 95 11692. Sources of hematopoietic stem cells include bone marrow cells, mobilized peripheral blood cells, and cord blood cells. In some embodiments, mobilized peripheral stem cells are used. In vitro expanded hematopoietic cells can also be used.

In some embodiments, the stem cells are from a stem cell bank, or are from a donor identified using a database of stem cell donors, e.g., a donor identified as having a immune profile that matches a tissue or organ to be transplanted. In some embodiments, the stem cells are from the stem cell, tissue, or organ donor.

In some embodiments, the present methods include the use of an allogeneic bone marrow inoculum that is not T cell-depleted. It has been suggested that “facilitator” T cells may contribute to the establishment of allogeneic hematopoietic chimerism (Schuchert et al., Nat. Med., 6:904-909, 2000; Kaufman et al., Blood, 84:2436-2446, 1994; and Fowler et al., Blood, 91:4045-4050, 1998). The primary reason for T cell depletion of donor bone marrow in human transplantation is to reduce the risk of GVHD. However, anti-CD154 mAb completely prevents GVHD in animal models (Sykes, Immunity, 14:417-424, 2001; Seung et al., Blood, 95:2175-2182, 2000; and Durie et al., J. Clin. Invest., 94:1333-1338, 1994). In other embodiments, the present methods include the use of allogeneic bone marrow that has been T-cell depleted, e.g., using methods known in the art, such as anti-T cell depleting antibodies plus complement or anti-T cell antibody coated magnetic bead separation methods.

V. Tissue and/or Organ Transplantation

The methods describe herein have a number of clinical applications. For example, the methods can be used in a wide variety of tissue and organ transplant procedures, e.g., the methods can be used to induce central tolerance in a recipient of a graft of stem cells such as bone marrow and/or of a tissue or organ such as pancreatic islets, liver, kidney, heart, lung, skin, muscle, neuronal tissue, stomach, and intestines. Thus, the new methods can be applied in treatments of diseases or conditions that entail stem cell tissue or organ transplantation (e.g., liver transplantation to treat hypercholesterolemia, transplantation of muscle cells to treat muscular dystrophy, or transplantation of neuronal tissue to treat Huntington's disease or Parkinson's disease). In some embodiments, the methods include administering to a subject in need of treatment: 1) a priming transfusion comprising allogeneic or xenogeneic cells that express donor or third party antigens, and/or a CD122 antagonist, e.g., an anti-CD122 antibody; 2) an antagonist of a molecule expressed on recipient T cells that mediates contact-dependent helper effector function, such as a CD154 antagonist (e.g., anti-CD154 antibody); 3) a stem cell transplant, e.g., bone marrow, and 4) a donor organ or tissue, e.g., liver, kidney, heart, lung, skin, muscle, neuronal tissue, stomach and intestines.

As described herein, the tissue or organ can be from the same donor as the hematopoietic stem cell donor and/or the donor of the priming transfusion, or a different donor. In some embodiments, one individual will donate the priming transfusion, the hematopoietic stem cells, and the tissue or organ. This will typically be the case where the donor is alive and viable, e.g., a volunteer donor of a regenerative or duplicated organ, e.g., a kidney, a portion of liver, or a bowel segment. In other embodiments, a first individual will donate the priming transfusion, and a second individual will donate the hematopoietic stem cells, and the tissue or organ. In some embodiments, a first individual will donate the priming transfusion, a second individual will donate the hematopoietic stem cells, and a third individual will donate the tissue or organ. This may more typically occur where the donors are, e.g., inbred animals, e.g., inbred pigs or non-human primates. In some embodiments, more than one individual will donate the stem cells, e.g., the population of stem cells will comprise cells from more than one donor.

In some embodiments, the transplanted tissue comprises pancreatic islets.

Accordingly, the invention encompasses a method for treating diabetes by pancreatic islet cell transplantation. The method comprises administering to a subject in need of treatment: 1) a priming transfusion comprising allogeneic or xenogeneic cells that express donor or third party antigens, and/or a CD122 antagonist, e.g., an anti-CD122 antibody; 2) an antagonist of a molecule expressed on recipient T cells that mediates contact-dependent helper effector function, such as a CD154 antagonist (e.g., anti-CD154 antibody); 3) a stem cell transplant, e.g., bone marrow; and 4) donor pancreatic islet cells. In some embodiments, the method further includes implanting an additional tissue or organ graft into the subject. Typically, the priming transfusion of allogeneic or xenogeneic cells, and at least one dose of the antagonist, are administered to the recipient prior to or simultaneously with administration of the bone marrow and the pancreatic islets.

In some embodiments, a donated tissue or organ is transplanted into the recipient once central tolerance has been established, e.g., about two weeks, about four weeks, about six weeks, about eight weeks, about ten weeks or more after a stem cell transplant, i.e., a bone marrow transplant, as described herein. Typically, the tissue or organ transplant will take place four to eight weeks after the stem cell transplant. Evidence of central tolerance includes the establishment of hematopoietic chimerism, e.g., at least about 0.5%, 1.0%, 1.5%, 2%, 5%, 10%, 15%, or more of circulating peripheral blood mononuclear cells are of donor origin. Any suitable method can be used to evaluate the establishment of chimerism. As one example, two color flow cytometry can be used, e.g., using monoclonal antibodies to distinguish between donor class I major histocompatibility antigens and leukocyte common antigens versus recipient class I major histocompatibility antigens. Alternatively chimerism can be evaluated by PCR. Tolerance to donor antigen can be evaluated by standard methods, e.g., by MLR assays.

In some embodiments, a donated tissue or organ is transplanted in a recipient concurrently with a stem cell transplant, i.e., a bone marrow transplant, as described herein. In some embodiments, the recipient is then treated with a regimen of immune-suppressing drugs to prevent rejection of the tissue or organ, e.g., until hematopoietic chimerism and central tolerance are established. Minimal regimens of immunosuppressive treatment are known, and one of skill in the art would appreciate that the regimen should be selected such that the regimen should be such that engraftment of the bone marrow transplant should not be undermined. For example, drugs such as rapamycin or cyclosporine A prevent costimulation-blockade induced tolerance. Again, any suitable method can be used to evaluate the establishment of chimerism. Tolerance to donor antigen can be evaluated by standard methods, e.g., by MLR assays. If natural anti-graft antibodies reappear before central tolerance is established, and if these antibodies cause damage to the donor tissue, the methods can be modified to permit sufficient time following BMT for central tolerance to be established prior to organ grafting.

In some embodiments, the donor is a living, viable human being, e.g., a volunteer donor, e.g., a relative of the recipient.

In some embodiments, the donor is no longer living, or is brain dead, e.g., has no brain stem activity. In some embodiments, the donor tissue or organ is cryopreserved.

In some embodiments, the donor is one or more non-human mammals, e.g., an inbred pig, or a non-human primate.

VI. Other Applications

In addition to their use in tissue and organ transplants, the new methods can be used to treat a wide variety of disorders. For example, the new methods can be used to treat autoimmune diseases. Lymphohemopoietic cells with abnormal function have been implicated in this class of disorders, and their replacement by cells derived from a new population of stem cells is a rational therapeutic approach. The reversal of these autoimmune diatheses by stem cell transplantation is likely to be associated with some degree of recovery in affected organ systems. For example, the present methods can be adapted to stem cell therapy protocols for the treatment of autoimmune disorders including, but not limited to, systemic lupus erythematosus, multiple sclerosis, rheumatoid arthritis, and scleroderma. A number of standard protocols are known, see, e.g., Sullivan and Furst, J. Rheumatol. Suppl., 48:1-4, 1997; Burt and Traynor, Curr. Op. Hematol., 5:472-7, 1998; Burt et al., Blood, 92(10):3505-14, 1998; Openshaw et al., Biol. Blood Marrow Transplant., 8:233-248, 2002. Accordingly, the invention includes methods for treating an autoimmune disorder, by administering to a subject in need of treatment: 1) a priming transfusion comprising allogeneic or xenogeneic cells that express donor or third party antigens; 2) an antagonist of a molecule expressed on recipient T cells that mediates contact-dependent helper effector function, such as a CD154 antagonist (e.g., anti-CD154 antibody); and 3) a stem cell transplant, e.g., bone marrow.

One of skill in the art will appreciate that the methods described herein can be adapted for the treatment of malignancy, e.g., hematological malignant disease. Immunocompetent donor cells, transplanted with the stem cells, have potent graft-versus-tumor activity (GVT) (see, e.g., Appelbaum, Nature, 411:385-389, 2001). The new methods provide (1) durable, sustained engraftment of stem cells without inducing GVHD, obviating the need for immunosuppression, and (2) donor-antigen specific transplant tolerance, thus preserving the potent GVT response. Thus, the new methods separate the GVT activity and GVHD activity, allowing the GVT response to be strengthened while avoiding GVHD, and are safer and far less toxic than conventional methods. Thus, the present invention includes methods of treating a subject having a hematologic malignancy, e.g., leukemia, by administering to the subject 1) a priming transfusion comprising allogeneic or xenogeneic cells that express donor or third party antigens; 2) an antagonist of a molecule expressed on recipient T cells that mediates contact-dependent helper effector function, such as a CD154 antagonist (e.g., anti-CD154 antibody); and 3) a stem cell transplant, e.g., bone marrow, under conditions suitable for the donor stem cells to exert a graft-versus-tumor effect.

The new methods can also be used to treat genetic disorders, e.g., hematologic disorders cause by a genetic mutation, such as beta-thalassemia and sickle cell. See, e.g., Yang and Hill, Pediatr. Infect. Dis. J., 20:889-900, 2001; and Persons and Nienhuis, Curr. Hematol. Rep., 2(4):348-55, 2003. Thus, the invention also includes methods for the treatment of a genetic disorder in a subject, by administering to the subject 1) a priming transfusion comprising allogeneic or xenogeneic cells that express donor or third party antigens; 2) an antagonist of a molecule expressed on recipient T cells that mediates contact-dependent helper effector function, such as a CD154 antagonist (e.g., anti-CD154 antibody); and 3) a stem cell transplant, e.g., bone marrow cells. In some embodiments, the cells of the stem cell transplant can be genetically modified, e.g., to express a particular protein that is useful in treating the genetic disorder. In some embodiments, the stem cells are from a donor who does not have the genetic disorder (e.g., normal stem cells), and the presence of the normal stem cells is sufficient to treat the genetic disorder.

The new methods can also be used to facilitate gene therapy (Bordignon and Roncarolo, Nat. Immunol., 3:318-321, 2002; Emery et al., Int. J. Hematol., 75:228-236, 2002; Park et al., Gene Ther., 9:613-624, 2002; Desnick and Astrin, Br. J. Haematol., 117:779-795, 2002; Bielorai et al., Isr. Med. Assoc. J., 4:648-652, 2002). Thus, in some embodiments, the stem cells are genetically altered, e.g., have at least one genetic modification, e.g., a modification that alters the expression of at least one gene, e.g., alters the level, timing, or localization of at least one gene.

### EXAMPLES

The invention is further described in the following examples, which do not limit the scope of the invention described in the claims.

### Methods

Animals

C57BL/6 (H2^{b}), BALB/c (H2^{d}), CBA/JCR (H2^{k}) and B10.BR (H2^{k}) mice of either sex were obtained from the National Cancer Institute, Frederick, Md. To investigate the fate of specific alloreactive CD8⁺ T cells, the KB5 TCR transgenic mouse was used, which has specificity to native H2b alloantigen (Tafuri et al., Science, 270:630-633, 1995; Kearney et al., Immunity, 1:327-339, 1994). This TCR transgenic mouse was the generous gift of Dr. John Iacomini (Harvard Medical School, Boston, Mass.) who obtained it from the original developer, Dr. Andrew Mellor (Medical College of Georgia, Augusta, Ga.). The TCR transgene is expressed by CD8⁺ cells in CBA (H2^{k}) mice and has specificity for H2-K^{b}. These transgenic T cells express a TCR that is recognized by the anti-clonotypic mAb DES (Tafuri et al., Science, 270:630-633, 1995).

All animals were certified to be free of Sendai virus, pneumonia virus of mice, murine hepatitis virus, minute virus of mice, ectromelia, LDH elevating virus, GD7 virus, Reo-3 virus, mouse adenovirus, lymphocytic choriomeningitis virus, polyoma, Mycoplasma pulmonis, and encephalitozoon cuniculi. Animals were housed in microisolator cages and given ad libitum access to autoclaved food and acidified water.

Antibodies and Flow Cytometry

FITC-conjugated anti-H2-K^{b} (clone AF6-88.5), PE-conjugated anti-H2-K^{k} (clone 36-7-5), and PE-conjugated anti-H2-K^{d} (clone SF1-1.1) monoclonal antibodies (mAbs) were obtained from PharMingen (San Diego, Calif.). MR1 hamster anti-mouse CD154 mAb was produced as ascites in scid mice and purified using a Protein A SEPHAROSE™ 4 Fast-flow purification column (Amersham Bioscienes, Piscataway, N.J.) and quantified by optical density (Iwakoshi et al., J. Immunol., 167:6623-6630, 2001; Noelle et al., Proc. Natl. Acad. Sci. U.S.A., 89:6550-6554, 1992). Antibody concentration was determined by measurement of optical density and confirmed by ELISA (Iwakoshi et al., J. Immunol., 167:6623-6630, 2001). The concentration of contaminating endotoxin was determined commercially (Charles River Endosafe, Charleston, S.C.) and was uniformly <10 units/mg of mAb (Iwakoshi et al., J. Immunol., 167:6623-6630, 2001).

Anti-CD4 (GK1.5), anti-CD8 (2.43) and anti-CD25 (PC61.5.3) antibodies were obtained from the American Type Culture Collection (Rockville, Md.). Antibodies for in vivo depletion were produced as ascites in scid mice and purified using a Protein G PLUS purification column (Oncogene Research Products, Boston, Mass.). To in vivo deplete CD4⁺ and CD8⁺ cells, mice were injected intraperitoneally with 0.5 mg of mAb on three consecutive days. To deplete CD25⁺ cells in vivo, mice were injected once intraperitoneally with 0.25 mg of the mAb. A hybridoma cell line secreting hamster anti-mouse CTLA4 mAb (clone 9H10) was the gift of Dr. James Allison (University of California, Berkeley, Calif.). Anti-CTLA4 mAb was grown as ascites, purified using a Protein A column (Oncogene Research Products, Boston, MA), and injected intraperitoneally at a dose of 0.075 mg per mouse daily on 3 consecutive days. The KB5-specific clonotypic DES antibody was produced from a mouse hybridoma cell line given to us by Dr. Iacomini. FITC-conjugated anti-mouse IgG2a developing reagent for DES (clone R19-15) was obtained from PharMingen.

Flow microfluorometry was performed as described (Forman et al., J. Immunol., 168:6047-6056. 2002; Seung et al., Blood 95:2175-2182, 2000; Iwakoshi et al., J. Immunol., 167:6623-6630, 2001). Briefly, 1×10⁶ viable cells were reacted with the appropriate antibody for 20 min at 4° C. In experiments using the KB5 synchimeras, cells were reacted with anti-DES antibody for 20 min at 4° C. Cells were then washed and reacted with FITC-conjugated anti-mouse IgG2a mAb (to develop the DES antibody). Whole blood was processed using BD FACS™ lysing solution (Becton Dickinson, San Jose, Calif.) in accordance with the protocol supplied by the manufacturer. Labeled cells were washed, fixed with 1% paraformaldehyde-PBS, and analyzed using a FACScan® fluorescence-activated cell-sorting instrument (Becton Dickinson, San Jose, Calif.). Lymphoid cells were gated according their light-scattering properties, and 30-50×10³ events were acquired for each analysis.

The relative percentages of host- and donor-origin cells in the various recipients of C57BL/6 (H2-K^{b+}) bone marrow were determined by flow microfluorometry. The percentage of peripheral blood mononuclear cells (PBMC) in chimeric mice expressing MHC class I was determined by dual labeling with anti-H2-K^{b} (donor) and anti-H2-K^{d} or anti-H2-K^{k} (recipient) antibodies. Because fewer than 100% of hematopoietic cells express MHC class I antigen, the relative percentage of donor-origin cells (H2-K^{b+}) in chimeric recipients was calculated as follows:

$$[ \frac{\% H2 - {K}^{b +}}{\% H2 - {K}^{b +} + \% H2 - {K}^{k + or d +}} ] \times 100 \%$$

In previous experiments, known mixtures of BALB/c and C57BL/6 peripheral blood mononuclear cells were analyzed, and it was determined that the lower limit of sensitivity of the assay for detecting either donor (H2-K^{b+}) or host (H2-K^{d+}) cells was 0.5% (Seung et al., Blood, 95:2175-2182, 2000).

Tolerance Induction and Bone Marrow Transplantation Procedures

Except as noted in specific experiments, bone marrow recipients were treated with our standard protocol for peripheral transplantation tolerance induction (Markees et al., J. Clin. Invest., 101:2446-2455, 1998; Iwakoshi et al., J. Immunol., 164:512-521, 2000; Iwakoshi et al., J. Immunol., 167:6623-6630, 2001). Relative to the transplantation of allogeneic bone marrow on day 0, mice received a single intravenous donor-specific transfusion (DST, 1×10⁷ spleen cells) on day −7 and four injections of MR1 anti-CD154 mAb (0.5 mg/dose) on days −7, −4, 0, and +3 (Markees et al., J. Clin. Invest., 101:2446-2455, 1998; Iwakoshi et al., J. Immunol., 164:512-521, 2000; Iwakoshi et al., J. Immunol., 167:6623-6630, 2001). The allograft consisted of 50×10⁶ or 100×10⁶ donor bone marrow cells in a volume of 0.5-1.0 ml injected via the lateral tail vein.

Donor mice were killed in 100% CO₂. For preparing the DST, spleens were removed, dispersed in sterile medium (RPMI-1640), washed, and counted. Cell viability was assayed by Trypan blue exclusion, and was >90% in all cases. The MR1 hamster anti-mouse CD154 mAb was produced as ascites in scid mice and purified as described (Forman et al., J. Immunol., 168:6047-6056. 2002; Iwakoshi et al., J. Immunol., 167:6623-6630, 2001; Foy et al., J.Exp.Med., 178:1567-1575, 1993). Bone marrow was obtained by flushing the femurs and tibias of donor mice with RPMI using a 24-gauge needle. Recovered cells were filtered through sterile nylon mesh (70 μm, Becton Dickinson, Franklin Lakes, N.J.), counted by hemocytometer, and re-suspended in RPMI.

Donor and recipient strain combinations are indicated in each Table in the examples below. Samples of peripheral venous blood were obtained from recipients at various intervals and the percentages of donor and host cells were determined by flow microfluorometry. Hematopoietic chimerism was defined as the presence of ≧0.5% donor-origin peripheral blood mononuclear cells.

Generation of KB5 TCR Transgenic Hematopoietic CBA Synchimeras

To examine the fate of both developing and mature alloreactive CD8⁺ T cells in a normal microenvironment, we used KB5 TCR transgenic hematopoietic chimeras (Iwakoshi et al., J. Immunol. 167:6623-6630, 2001). The TCR transgene is expressed by CD8⁺ cells in CBA (H₂^{k}) mice and has specificity for H2-K^{b}. Small numbers of KB5 transgenic bone marrow cells were injected into sub-lethally irradiated syngeneic CBA non-transgenic hosts, to generate as “synchimeric” mice. In this system, the mice circulate a self-renewing trace population of anti-H2-K^{b} alloreactive CD8⁺ T cells maturing in a normal microenvironment (Iwakoshi et al., J. Immunol. 167:6623-6630, 2001).

The synchimeras were generated as described (Iwakoshi et al., J. Immunol., 167:6623-6630, 2001). Briefly, bone marrow cells were collected as described above from male and female KB5×CBA/JCr/F1 mice (H2^{k}). Recipients were male CBA/JCr mice 4-7 weeks of age treated with 2 Gy whole body gamma irradiation using a ¹³⁷Cs source (Gammacell 40, Atomic Energy of Canada, Ottawa, ON, Canada). They were then injected intravenously with 0.5×10⁶ transgenic bone marrow cells in a volume of 0.5 ml via the lateral tail vein within 2-5 hours of irradiation. The transgenic T cells that develop express an anti-H2-K^{b} specific TCR recognized by the mAb DES (Tafuri et al., Science, 270:630-633, 1995). These procedures have been documented to generate a stable population of DES⁺CD8⁺ cells that comprise 5-8% of PBMC within 8 weeks of bone marrow transplantation (Iwakoshi et al., J. Immunol., 167:6623-6630, 2001).

Skin Transplantation

Full-thickness skin grafts approximately 1 cm in diameter were obtained from shaved euthanized donors, scraped to remove muscle, and grafted without suturing onto prepared sites on the flanks of anesthetized recipients as described (Markees et al., J. Clin. Invest., 101:2446-2455, 1998). Skin grafts were dressed with Vaseline™-impregnated gauze and an adhesive bandage for the first week after surgery. Thereafter, skin grafts were assessed 3 times weekly, and rejection was defined as the first day on which the entire graft surface appeared necrotic (Markees et al., J. Clin. Invest., 101:2446-2455, 1998).

Statistical Analysis

Parametric data are presented as the arithmetic mean±1 s.d. Comparisons of three or more means used oneway analyses of variance and the least significant difference procedure for a posteriori contrasts (Nie et al., Statistical Package for the Social Sciences. McGraw-Hill, New York., pp. 1-675 1975). Comparisons of two means used unpaired t-tests without assuming equal variance (Glantz, Primer of Biostatistics. McGraw-Hill, New York. pp. 352, 1991). In experiments in which large variances were observed, groups were compared non-parametrically with the Mann-Whitney U or Kruskall-Wallis tests (Siegel, Nonparametric Statistics. McGraw-Hill, New York. pp. 1-239, 1956). Analysis of contingency tables used the ² statistic or, in the case of 2×2 tables, the Fisher exact statistic (Siegel, Nonparametric Statistics. McGraw-Hill, New York. pp. 1-239, 1956). Skin allograft survival among groups was compared using the method of Kaplan and Meier (Kaplan and Meier, J. Am. Statist. Assn., 53:457-481, 1958); the equality of allograft survival distributions for animals in different treatment groups was tested using the log rank statistic (Matthews and Farewell, eds., The Log-Rank or Mantel-Haenszel Test for the Comparison of Survival Curves in Using and Understanding Medical Statistics. Karger, Basel. pp. 79-87, 1988). P values <0.05 were considered statistically significant.

### Example 1

### Peripheral Transplantation Tolerance Induction Facilitates Allogeneic Stem Cell Engraftment

1.1 Establishment of allogeneic hematopoietic chimerism in the absence of host myeloablative conditioning in BALB/c mice

The peripheral transplantation tolerance-induction protocol described herein was first evaluated for its ability to facilitate the generation of hematopoietic chimerism. Briefly, BALB/c (H2^{d}), CBA/J (H2^{k}) or B10.BR (H2^{k}) mice were randomized to the indicated treatment groups and injected with C57BL/6 (H2^{b}) bone marrow cells at the dose indicated on day 0. Mice treated with a donor-specific transfusion (DST) received 10⁷ C57BL/6 spleen cells on day −7 relative to bone marrow transplantation. Mice treated with anti-CD154 mAb received 4 doses of 0.5 mg intraperitoneally on days −7, −4, 0, and +3. Hematopoietic chimerism was defined as the presence of ≧0.5% donor-origin (H2-K^{b+}) PBMC 6 to 9 weeks after transplantation as described herein. As shown in Table 1 (Group 1), 89% of treated BALB/c mice became chimeric. The percentage of donor-origin PBMC in these mice 8 to 9 weeks after bone marrow transplantation averaged ˜9%. In contrast, in the absence of DST treatment there was no evidence of chimerism in any BALB/c mice treated with bone marrow and anti-CD154 mAb (Table 1, Group 7).

TABLE 1
Hematopoietic Chimerism in Recipients of C57BL/6 Bone Marrow

| | | | | | | | Donor |
|---|---|---|---|---|---|---|---|
| | | | | | | | Origin |
| | | | | | Bone | Frequency of | PBMC in |
| | | | | Anti- | Marrow | Hematopoietic | Hem- |
| | | Myeloablative | | CD154 | Dose | Chimerism | Chimeric |
| Group | Recipient | Conditioning | DST | mAb | (×10⁶) | (%) | Mice (%) |
| 1 | BALB/c | No | Yes | Yes | 50 | 17/19 (89%)^{a} | 9.2 ± 3.2 |
| 2 | CBA/J | No | Yes | Yes | 50 | 17/25 (68%) | 8.3 ± 4.5 |
| 3 | B10.BR | No | Yes | Yes | 50 | 14/14 (100%) | 16.9 ± 12.4^{b} |
| 4 | BALB/c | Yes | Yes | Yes | 50 | 5/5 (100%) | 37.2 ± 4.3%^{c} |
| 5 | CBA/J | Yes | Yes | Yes | 50 | 9/9 (100%) | 35.6 ± 5.2%^{d} |
| 6 | CBA/J | No | Yes | Yes | 100 | 5/5 (100%) | 16.5 ± 5.7%^{d} |
| 7 | BALB/c | No | No | Yes | 50 | 0/10 (0%)* | — |
| 8 | CBA/J | No | No | Yes | 50 | 0/9 (0%)* | — |
| 9 | B10.BR | No | No | Yes | 50 | 15/15 (100%) | 19.7 ± 10.9 |
| 10 | B10.BR | No | No | No | 50 | 0/8 (0%)* | — |

*The percentage of donor-origin PBMC in non-chimeric mice was in all cases below the limit of detection (<0.5%).
^{a}p = N.S. vs. Groups 2 and 3.
^{b}p < 0.01 vs. groups 1 and 2.
^{c}p < 0.01 vs. Group 1.
^{d}p < 0.01 vs. Group 2.

To assess the durability and variability of chimerism, PBMC were re-measured at intervals 4 to 30 weeks after transplantation in two independent cohorts of chimeric mice. In independent trials, two groups of BALB/c (H2d) mice (N=5 in each group) were injected with C57BL/6 (H2b) bone marrow cells (50×106) on day 0. All mice were treated with a donor-specific transfusion consisting of 107 C57BL/6 spleen cells on day −7 relative to bone marrow transplantation. They also received anti-CD154 mAb at a dose of 0.5 mg intraperitoneally on days −7, −4, 0, and +3 relative to bone marrow transplantation. The percentage of donor-origin peripheral blood mononuclear cells (PBMC) was measured by flow microfluorometry in all mice 4, 8, 14, or 21, and 30 weeks after bone marrow transplantation as described herein.

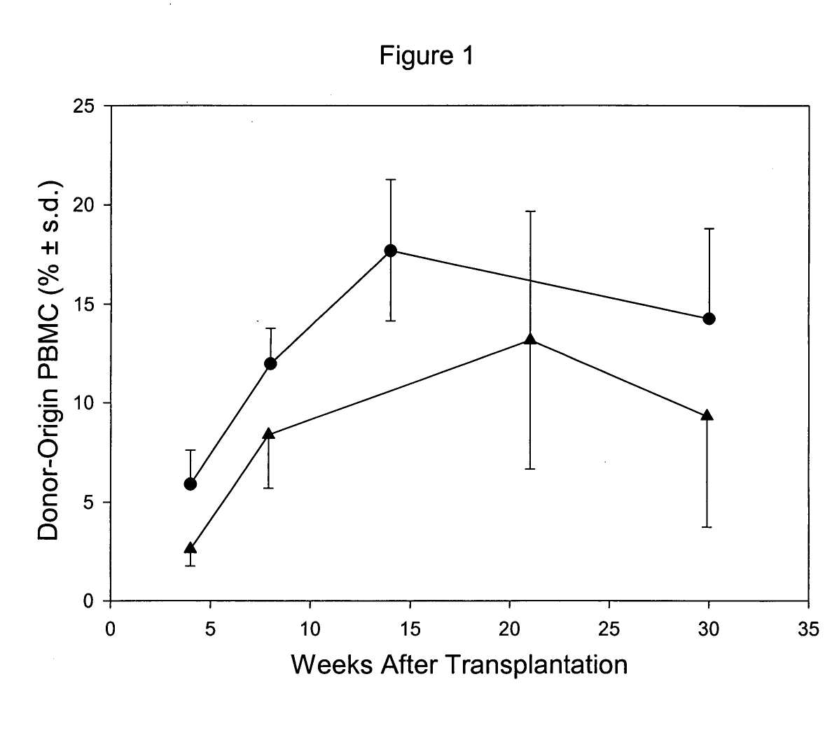

Chimerism was readily detectable 4 weeks after transplantation and rose to ˜10% (range 6 to 15%, N=10) by week 8 (FIG. 1). At 30 weeks after transplantation, all mice remained chimeric and the levels of chimerism were similar to those at week 8 (˜12%, range 2 to 20%, FIG. 1). Thus, the methods described herein can be used to generate durable allogenic hematopoietic chimerism in the absence of myeloablative host conditioning.

1.2 The Levels of Hematopoietic Chimerism Achieved Generate Donor-Specific Transplantation Tolerance in the Absence of GVHD

Although allogeneic hematopoietic chimerism was achieved, the levels of chimerism were relatively low, e.g., typically less than about 10%. To evaluate the sufficiency of these levels to generate transplantation tolerance, randomly subsets of both chimeric and non-chimeric BALB/c mice from Table 1 (Groups 1 and 7) were transplanted with C57BL/6 skin allografts 9 weeks after injection of C57BL/6 bone marrow selected as described in Methods. Mice were observed through day 142 after skin transplantation. Median survival time (MST) of skin allografts in the chimeric mice was >96 days (Table 2). In contrast, most non-chimeric mice rejected skin allografts rapidly (MST=11 days, p <0.0005). To document that this state of transplantation tolerance was donor-specific, additional chimeric BALB/c mice were transplanted 9 weeks after bone marrow transplantation with third-party skin allografts from CBA/J donors. Survival of these four allografts was very brief, all of them rejecting by day 14 (Table 2, p=N.S. vs. non-chimeric mice).

Animals were observed for signs of GVHD throughout the period of observation (up to 30 weeks). There was no sign of illness in any chimeric bone marrow recipient given the anti-CD154 mAb regimen. Thirty weeks after transplantation, four chimeric BALB/c mice were selected at random and studied histologically. There was no evidence of GVHD in samples of skin, liver, or small or large intestine in any of the mice. Thus, the levels of chimerism achieved were sufficient to generate central transplant tolerance. This effect was bone-marrow donor-specific, and no GVHD was observed.

TABLE 2
Duration of Allogeneic Skin Graft Survival

| | | Bone | | Skin | | |
|---|---|---|---|---|---|---|
| | DST | Marrow | | Allograft | MST | Skin Allograft |
| Host | Donor | Donor | Chimeric | Donor | (Days) | Survival (Days) |
| BALB/c | C57BL/6 | C57BL/6 | Yes | C57BL/6 | >96 | 38, >56, 80, 80, >112, |
| | | | | | | >112, >142, >142^{a} |
| BALB/c | C57BL/6 | C57BL/6 | Yes | CBA/J | 14 | 14, 14, 14, 14 |
| BALB/c | — | C57BL/6 | No | C57BL/6 | 11 | 11, 11, 11, 11, 49 |

MST: median survival time.
^{a}p < 0.0005 vs. other groups.

1.3 Allogeneic Stem Cell Engraftment Using anti-CD154 mAb and DST but no Myeloablation can be achieved in CBA/J and B 10.BR Recipient Mice

To determine if the engraftment of allogeneic bone marrow cells in mice treated with DST plus anti-CD154 mAb is strain-dependent, the same experiment was performed using two different strains of mice as recipients. When tested 8 to 9 weeks after administration of DST, anti-CD154 mAb, and C57BL/6 bone marrow cells, 68% of CBA/J mice (Table 1, Group 2) and 100% of B10.BR mice (Group 3) had become chimeric. In both cases, the frequency of chimerism was statistically similar to that achieved using BALB/c recipients (Group 1, p=N.S.). The percentage of donor-origin PBMCs in chimeric CBA/J mice (˜8%) was similar to that in chimeric BALB/c mice (p=N.S.) but levels in both BALB/c and CBA recipients were significantly less (p<0.01) than levels achieved in B10.BR mice (˜17%).

Like BALB/c mice, CBA/J recipients of bone marrow and anti-CD154 mAb but no DST did not become chimeric (Table 1, Group 8). In contrast, B10.BR mice treated in the same way uniformly became chimeric (Group 9). As was true for B10.BR recipients given both anti-CD154 mAb and a DST (Group 3), 20% of their PBMC were of donor-origin. In both groups of B10.BR chimeras, the percentage of donor-origin cells was quite variable, ranging from 2.6% to 46.0%. B10.BR mice treated with a bone marrow graft but neither anti-CD154 mAb nor DST failed to become chimeric (Group 10). Thus, the engraftment of stem cells in mice treated with a priming transfusion plus anti-CD154 mAb is not strain-specific.

1.4 Increasing Bone Marrow Cell Dose or Adding Minimal Myeloablative Conditioning Increases Levels of Chimerism

Because hematopoietic chimerism can be established in the absence of myeloablative conditioning if very large numbers of bone marrow cells are transplanted (Durham et al., J. Immunol., 165:1-4, 2000; Wekerle et al., Nat. Med., 6:464-469, 2000), the effect of increasing the donor inoculum in mice treated with both DST and anti-CD154 mAb was evaluated. Transplantation of 100×10⁶ C57BL/6 bone marrow cells into CBA/J recipients was associated with uniform generation of chimerism (Table 1, Group 6), and the percentage of donor-origin PBMC in these mice was, on average, double that observed in CBA/J recipients of 50×10⁶ C57BL/6 cells (Table 1, Group 2, p=0.025).

The addition of minimal myeloablation also appeared to improve outcome. Both BALB/c (Table 1, Group 4) and CBA/J (Group 5) recipients uniformly became chimeric if treated with 1 Gy of whole body irradiation prior to DST, anti-CD154 mAb, and infusion of 50×10⁶ C57BL/6 bone marrow cells. In both cases, donor-origin cells comprised more than a third of the PBMC population 6-7 weeks after bone marrow injection, and these percentages were statistically significantly greater than the percentages achieved without conditioning (Table 1, Groups 1 and 2, p<0.001 for both comparisons). Thus, the use of larger doses of bone marrow cells, and/or the use of minimal myeloablative host conditioning, increases the success rate for the generation of hematopoietic chimerism.

### Example 2

### Timing of DST and anti-CD154 mAb Treatment is Important for Generation of Allogeneic Chimerism

Studies of solid organ transplantation tolerance induction have shown that administration of DST plus anti-CD154 mAb leads to the deletion of peripheral host alloreactive CD8⁺ T cells, an effect that is maximal 3 days after the initiation of treatment (Iwakoshi et al., J. Immunol., 164:512-521, 2000). To test the hypothesis that deletion of host alloreactive CD8⁺ T cells would define the optimal time point at which allogeneic bone marrow chimerism could be achieved in the absence of myeloablative conditioning, the timing of DST plus anti-CD154 mAb treatment in relation to C57BL/6 bone marrow transplantation into CBA/J recipients was varied. Briefly, groups of CBA/J (H2k) mice were randomized and transplanted with 50×106 C57BL/6 (H2b) bone marrow cells on day 0. All mice also received a single C57BL/6 DST consisting of 107 spleen cells on days −3, −5, −10, or −14 relative to bone marrow transplantation. In addition, all mice were injected intraperitoneally with 4 doses of 0.5 mg anti-CD154 mAb on days 0, +3, +7, and +10 relative to the DST. The temporal relationship of the DST and anti-CD154 mAb injections was the same as in Table 1; only the timing of the bone marrow graft was varied. No myeloablative conditioning was performed. Chimerism was defined as the presence of ≧0.5% donor-origin (H2-Kb+) PBMC 6 weeks after transplantation. In these experiments, the first of the four injections of anti-CD154 mAb was always given immediately before the DST.

When DST was given 10 or 14 days before bone marrow transplantation, 60% and 80% of recipients, respectively, became chimeric (Table 3, Groups 1 and 2); this rate of success was comparable to that achieved when DST was injected 7 days before transplantation (68%, Table 1, Group 2, χ²=0.44, p=N.S.). The percentage of donor-origin PBMC detected 6 or more weeks after transplantation in the mice that became chimeric was ˜10%, irrespective of the timing of the DST.

In contrast, when DST was injected 5 or 3 days before bone marrow transplantation, no recipients became chimeric (Table 3, Groups 3 and 4, χ²=13.22, p<0.02 vs. Table 1, Group 2). Given that host alloreactive CD8⁺ T cells are believed to be deleted in mice treated with DST plus anti-CD154 mAb at these time points (Iwakoshi et al., J. Immunol., 164:512-521, 2000), the result was unexpected.

TABLE 3
Hematopoietic Chimerism in CBA/J Recipients of C57BL/6 Bone Marrow

| | | | Percentage of |
|---|---|---|---|
| | Day of DST | Frequency of | Donor Origin PBMC |
| Group | Injection | Chimerism (%) | in Chimeric Mice (%) |
| 1 | −14 | 4/5 (80%)^{a} | 10.2 ± 1.9 |
| 2 | −10 | 3/5 (60%)^{a} | 10.4 ± 4.2 |
| 3 | −5 | 0/5 (0%)^{b} | — |
| 4 | −3 | 0/5 (0%)^{b} | — |

^{a}p = N.S. vs. Table 1 Group 2.
^{b}p < 0.01 vs. Table 1 Group 2.

Thus, optimal timing of the bone marrow transplantation is more than five days after the priming transfusion, e.g., at least six, seven, eight, nine, ten, eleven, twelve, thirteen, fourteen or more days later.

### Example 3

### The Combination of DST, Anti-CD154 mAb, and Bone Marrow Engraftment Leads to Permanent Deletion of Host Alloreactive CD8⁺ Peripheral T Cells

Studies of peripheral tolerance induction using DST plus anti-CD154 mAb have documented that after host alloreactive CD8⁺ T cells are deleted, the cells reappear over time, and their reappearance is associated with rejection of healed-in allografts (Iwakoshi et al., J. Immunol., 167:6623-6630, 2001). Given the apparent permanence of hematopoietic chimerism in mice treated with DST, anti-CD154 mAb, and bone marrow, it is possible that establishment of chimerism would lead to permanent deletion of peripheral alloreactive CD8⁺ T cells. To test this hypothesis, KB5 synchimeric mice were used. These mice circulate small numbers of TCR transgenic alloreactive CD8⁺ T cells that are continuously replenished over time as newly generated KB5 T cells are released from the thymus (Iwakoshi et al., J. Immunol., 167:6623-6630, 2001).

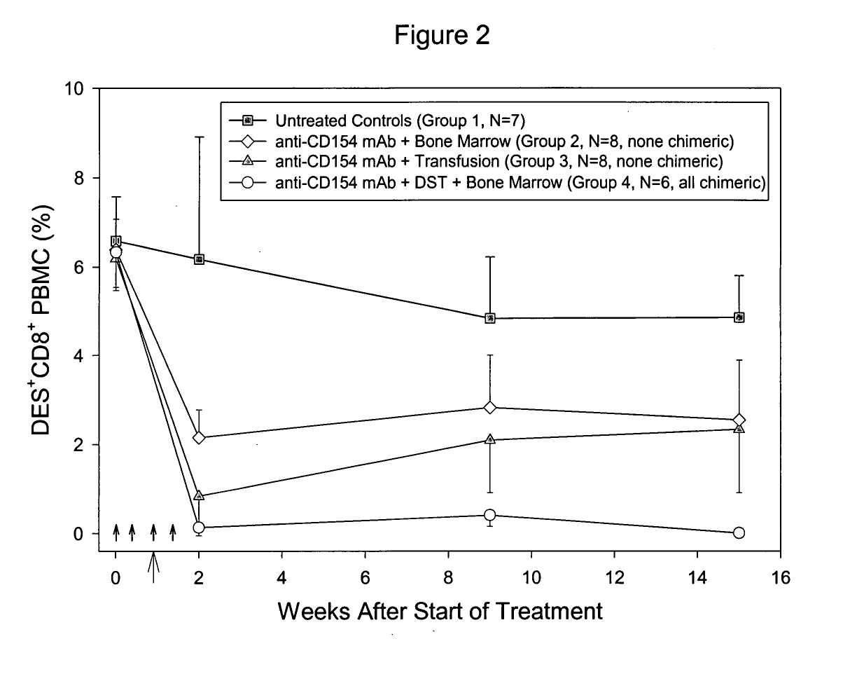

KB5 CBA synchimeric mice were randomized into 4 groups. Mice in group 1 were untreated. Mice in Group 2 were injected with 4 doses of anti-CD154 mAb on days 0, +3, +7, and +10 (FIG. 2, small arrows) relative to injection of 50×106 C57BL/6 bone marrow cells on day +7 (long arrow). Group 3 received 4 doses of 0.5 mg of anti-CD154 mAb at the same intervals (small arrows) plus a transfusion of C57BL/6 spleen cells on day 0. Group 4 received a donor-specific transfusion of C57BL/6 spleen cells on day 0 and anti-CD154 mAb on days 0, +3, +7, and +10 relative to injection of 50×106 C57BL/6 bone marrow cells on day 7. The percentage of DES⁺CD8⁺ cells in the blood was determined on day 0 (before any treatment) and then at the indicated times. Within two weeks of treatment, the percentage of DES⁺CD8⁺ peripheral blood cells was significantly lower in all treatment groups compared with controls (p<0.001). Thereafter, the percentage of DES⁺CD8⁺ peripheral blood cells in Groups 2 and 3 tended to rise towards that observed in controls, but even at week 15 the percentage remained less than in controls (p<0.001). In contrast, the percentage of DES⁺CD8⁺ peripheral blood cells in Group 4 remained extremely low throughout the course of the experiment, and at week 15 was significantly lower than in all other groups (pL0.001 for each comparison). With respect to chimerism, defined as >0.5% donor-origin (H2-Kb+) PBMC 9 weeks after transplantation, all mice in group 4 were chimeric and none in Groups 2 or 3 were chimeric.

The level of alloreactive DES⁺CD8⁺ T cells in the peripheral blood of these 4 groups of mice is shown in FIG. 2. The level of DES⁺CD8⁺ T cells in control mice during the period of observation was ˜5% to ˜6.5%; these levels are comparable to those we have reported previously in this model system (Iwakoshi et al., J. Immunol., 167:6623-6630, 2001).

None of the mice in Groups 2 or 3 became chimeric; no donor-origin cells were detectable at any time point throughout the 15 week period of observation. As expected, and consistent with previous reports (Iwakoshi et al., J. Immunol., 167:6623-6630, 2001), the level of alloreactive DES+CD8⁺ T cells in mice treated with anti-CD154 mAb and a C57BL/6 splenocyte transfusion (Group 3) was much lower within two weeks of transfusion (˜0.8%). Thereafter the levels rose slowly and recovered to ˜2.4% by week 15. The behavior of mice treated with anti-CD154 mAb and bone marrow (Group 2) was similar, although the initial decline was less dramatic than that associated with the use of a splenocyte transfusion alone.

The results for the mice treated with anti-CD154 mAb, a splenocyte DST, and then with a bone marrow allograft (Group 4) were much different. As expected, 6 of 8 (75%) became chimeric. The percentage of donor-origin PBMC 9 weeks after transplantation was high (22.3+13.2%) and remained at about this level throughout the 15 week period of observation. In striking contrast to the outcome in the other Groups, the level of DES⁺CD8⁺ T cells in the six chimeric mice fell within 2 weeks to levels that were below the limit of detection and remained at that low level throughout the period of observation (p≦0.001 vs. all other groups at week 15). Thus, establishment of chimerism using the methods described herein can lead to long-term deletion of peripheral alloreactive CD8+ T cells.

### Example 4

### Intrathymic Deletion of Alloreactive DES⁺CD8⁺CD4⁻ T Cells in Allogeneic Hematopoietic Chimeras

4.1 Normal Distribution of CD4 and CD8 Cells in the Thymus of KB5 Synchimeric Mice

The long-term absence of peripheral alloreactive DES⁺CD8⁺ cells in mice with hematopoietic chimerism suggested that they might be undergoing intrathymic deletion. Before proceeding to test this possibility, overall thymic maturation was analyzed in the untreated KB5 synchimeric mouse. KB5 transgenic mice used to generate synchimeras have an abnormally large population of single-positive CD8⁺ cells in the thymus (Manilay et al., Transplantation, 66:96-102, 1998). However, KB5 synchimeric mice exhibit a normal distribution of total CD4⁺ and CD8⁺ thymocytes (Mathieson and Fowlkes, Immunol. Rev., 82:141-173, 1984; Shortman et al., Curr. Top. Microbiol. Immunol., 126:5-18, 1986; Ceredig and Cummings, J. Immunol., 130:33-37, 1983).

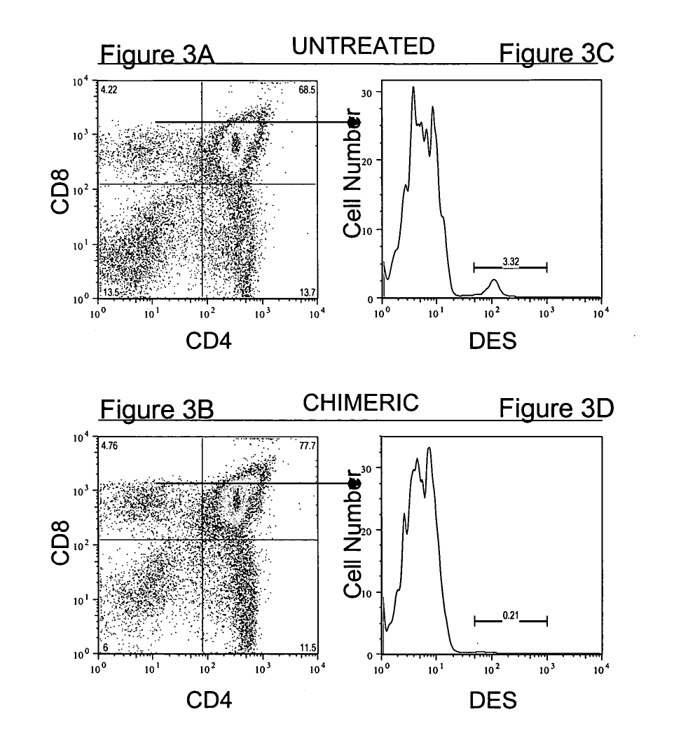

Intrathymic deletion of host alloreactive DES+CD8+CD4-thymocytes. KB5 CBA synchimeras were randomized into 2 groups. Group 1 (upper panels) was left untreated. Group 2 (lower panels) was injected with a C57BL/6 DST on day -7 and anti-CD154 mAb on days −7, −4, 0, and +3 relative to injection of 50×106 C57BL/6 bone marrow cells on day 0. Thymi were recovered 35 weeks after bone marrow transplantation and analyzed by flow microfluorometry for the percentage of DES+CD8+CD4-thymocytes as described herein. Shown in the left column are representative dot plots; the percentage of cells expressing CD4 and CD8 is indicated in each quadrant. The right column presents histograms; the horizontal bars depict the gates used to determine the number of DES+ cells in the CD8+CD4-quadrant. FIGS. 3A-3D illustrate representative data; the complete dataset is given in Table 4.

TABLE 4
Hematopoietic Chimerism and Host Alloreactive CD8⁺ T Cells
in KB5 Synchimeric Recipients of C57BL/6 Bone Marrow

| | | | | | | Host |
|---|---|---|---|---|---|---|
| | | | | | Time of Analysis | CD8⁺CD4⁻ |
| | | Bone | | | Relative to | Thymocytes |
| | Transfusion | Marrow | | | completion of | that were DES⁺ |
| Group | Donor | Donor | N | Chimeric | treatment (Days) | (%) |
| 1 | None | None | 11 | — | Same | 26.4 ± 20.8^{a} |
| | | | | | approximate age | |
| | | | | | as groups 2-8; | |
| | | | | | No treatment | |
| | | | | | given | |
| 2 | C57BL/6 | None | 3 | — | −3 d | 18.6 ± 4.8 |
| 3 | C57BL/6 | None | 7 | — | 0 d | 25.7 ± 11.2 |
| 4 | C57BL/6 | None | 4 | — | +15 d | 38.2 ± 21.7 |
| 5 | C57BL/6 | C57BL/6 | 4 | *See | +15 d | 16.2 ± 13.8^{b} |
| | | | | legend | | |
| 6 | C57BL/6 | None | 6 | No | +21-30 d | 30.4 ± 13.9 |
| 7 | C57BL/6 | C57BL/6 | 3 | No | +21-30 d | 30.4 ± 8.9 |
| 8 | C57BL/6 | C57BL/6 | 3 | Yes | +21-30 d | All < 0.2^{c} |
| 9 | None | None | 3 | — | Age matched, | 2.8 ± 0.6 |
| | | | | | never treated | |
| 10 | C57BL/6 | C57BL/6 | 2 | No | 35 weeks | 4.4, 18.3 |
| 11 | C57BL/6 | C57BL/6 | 3 | Yes | 35 weeks | All < 0.2^{d} |

^{a}p = N.S. vs. groups 2 and 3.
^{b}p = 0.08 vs. group 4.
^{c}p < 0.01 vs. groups 6 and 7.
^{d}p < 0.05 vs. combined groups 9 and 10.
*At the 15 day time point, it cannot reliably be determined if mice are chimeric.

The thymocytes of untreated KB5 synchimeric mice consisted of a large population of double-positive cells (81.5±4.3%, N=14) and smaller populations of CD4⁺CD8⁻ single positive cells (9.4±1.4%, N=14) and CD4⁻CD8⁺ (4.4±16%, N=14) single positive cells. Representative histograms are shown in FIG. 3. These percentages of single and double positive thymocytes are typical of those observed in normal untreated adult mice (Mathieson and Fowlkes, Immunol. Rev., 82:141-173, 1984; Shortman et al., Curr. Top. Microbiol. Immunol., 126:5-18, 1986; Ceredig and Cummings, J. Immunol., 130:33-37, 1983). Thus, the methods described herein lead to a durable central tolerance, with intrathymic deletion of alloreactive thymocytes.

4.2 DES⁺CD8⁺CD4⁻ Thymocytes are not deleted by Treatment with DST plus Anti-CD154 mAb

Having determined that the overall distribution of thymocyte CD4⁺ and CD8⁺ phenotypes in synchimeric mice is normal, the percentages of DES⁺CD8⁺ thymocytes following costimulation blockade and splenocyte transfusion was measured. Before any treatment, the percentage of CD8⁺CD4⁻ thymocytes that were also DES+was 26.4±20.8% (Table 4, Group 1). DES⁺CD8⁺CD4⁻ thymocytes were also readily detectable at statistically similar levels 4 and 7 days after treatment with DST and anti-CD154 mAb (Table 4, Groups 2 and 3, p=N.S.). In contrast, it is known that that peripheral DES⁺CD8⁺ cells are deleted within 3 days of treatment with DST plus anti-CD154 mAb, well before graft placement (Iwakoshi et al., J. Immunol. 164:512-521, 2000).

4.3 Bone Marrow Cell Engraftment in Mice Treated with DST plus Anti-CD154 mAb Leads to Intrathymic Deletion of DES⁺CD8⁺CD4⁻ Cells

We next tested the hypothesis that successful generation of hematopoietic chimerism subsequent to treatment with DST and anti-CD154 mAb would lead to the deletion of DES⁺CD8⁺CD4⁻ alloreactive thymocytes. Briefly, CBA/J (H2^{k}) mice of about four weeks of age were irradiated (2 Gy) and injected with bone marrow from KB5 CBA/J TCR transgenic donors as described in Methods. Eight to 10 weeks later, with no additional irradiation, these KB5 CBA/J synchimeras received a donor-specific transfusion consisting of 10⁷ C57BU6 (H2^{b} ) spleen cells on day −7 plus 4 intraperitoneal doses of anti-CD154 mAb (0.5 mg/dose) on days −7, 4, 0, +3 relative to intravenous injection of 50×10⁶ C57BL/6 bone marrow cells on day 0. Thymi were recovered at the indicated time points relative to marrow transplantation on day 0 and the percentage of host anti-donor alloreactive DES³⁰ CD8⁺CD4⁻ thymocytes was measured by flow microfluorometry.

As shown in Table 4, 15 days after bone marrow transplantation the percentage of DES⁺CD8⁺CD4⁻ thymocytes in mice treated with DST and anti-CD154 mAb (Table 4, Group 5) was ˜50% less than in age-matched mice that had been given DST and anti-CD154 mAb but no graft (Group 4), but at this time point the difference was not statistically significant (p=0.08). By 21-30 days after bone marrow injection, it was possible to distinguish chimeric and non-chimeric recipients. At this time point levels of DES⁺CD8⁺CD4⁻ thymocytes remained at high baseline levels in both mice that had received DST plus anti-CD154 mAb but no graft (Table 4, Group 6) and in mice that had received DST plus anti-CD154 mAb plus a graft but had not become chimeric (Group 7). In contrast, DES⁺CD8⁺CD4⁻ thymocytes were undetectable (<0.3%) in all chimeric mice (Group 8, p<0.01 vs. both Groups 6 and 7).

Additional mice were studied 35 weeks after treatment to assess the durability of alloreactive thymocyte deletion. We first noted that the percentage of DES⁺CD8⁺CD4⁻ thymocytes in age-matched but untreated synchimeras had spontaneously fallen over time, but were nonetheless readily detectable. The decline was from ˜26% at baseline (Table 4, Group 1) to ˜3% 8 to 9 months later (Table 4, Group 9, p=0.01). DES⁺CD8⁺CD4⁻ thymocytes were also readily detectable in two recipients of DST, anti-CD154 mAb, and bone marrow that had not become chimeric (Group 10). In contrast, no DES⁺CD8⁺CD4⁻ thymocytes could be detected in any of three recipients of DST, anti-CD154 mAb, and bone marrow that had become and remained chimeric (Table 4, Group 11, p<0.05). A representative histogram documenting the disappearance of DES⁺CD8⁺CD4⁻ thymocytes in one of the chimeric mice from Group 11 is shown in FIG. 3. Although lacking in DES⁺CD8⁺CD4⁻ thymocytes, the thymi of these mice 35 weeks after bone marrow transplantation were clearly allochimeric. The presence of both host (H2-K^{k}) and donor (H2-K^{b}) thymocytes was confirmed by flow microfluorometry.

### Example 5

### Allospecific and Non-Allospecific Mechanisms are Important for Engraftment of Bone Marrow Cells

5.1 MHC-Matching of the DST and Bone Marrow Donors is not Required for Allogeneic Bone Marrow Engraftment in Normal Mice

Induction of peripheral transplantation tolerance using DST and anti-CD154 mAb requires that the MHC of the transfusion donor be the same (i.e., “donor specific”) as that of the graft donor (Markees et al., J. Clin. Invest., 101:2446-2455, 1998). To evaluate whether successful generation of hematopoietic chimerism using the protocols described herein would also require MHC-matching of the transfusion and bone marrow cells, i.e. “donor-specific” transfusion, CBA mice (H2^{k}) were treated with a C57BL/6 (H₂^{b}) non-donor specific spleen cell transfusion plus anti-CD154 mAb and then injected with BALB/c (H2^{d}) bone marrow cells. Briefly, CBA/J (H2k) and KB5 CBA/J TCR transgenic synchimeric mice (H2k) were injected intravenously with 107 spleen cells on day −7 and intraperitoneally with 4 doses of 0.5 mg anti-CD154 mAb on days −7, −4, 0, +3 relative to intravenous injection of 50×106 bone marrow cells on day 0. Transfusion and bone marrow donors were either C57BL/6 (H2b) or BALB/c (H2d) as indicated. No myeloablative conditioning was used. The percentage of donor-origin PBMC was measured 8-9 weeks after bone marrow transplantation by flow microfluorometry. Chimerism was defined as the presence of 30.5% PBMC of donor-origin. a: p<0.01 vs. group 4. Unexpectedly, all became chimeric (Table 5, Group 1). To verify this unexpected outcome, we reversed the DST and bone marrow donors. CBA mice (H₂^{k}) were treated with BALB/c (H2^{d}) spleen cell transfusion plus anti-CD154 mAb and then injected with C57BL/6 (H2^{b}) bone marrow cells. Again, the majority (90%) of these mice became chimeric (Table 5, Group 2).

TABLE 5
Frequency of Chimerism in CBA/J and KB5 CBA/J Bone Marrow Recipients

| | | | | | Percentage |
|---|---|---|---|---|---|
| | | | | | of Donor Origin |
| | | | | Frequency of | PBMC in Hem- |
| | | Transfusion | Bone Marrow | Chimerism | Chimeric Mice |
| Group | Host | Donor | Donor | (%) | (%) |
| 1 | CBA/J | C57BL/6 | BALB/c | 9/9 (100%) | 2.8 ± 0.8 |
| 2 | CBA/J | BALB/c | C57BL/6 | 9/10 (90%) | 7.4 ± 2.1 |
| 3 | KB5 CBA/J | C57BL/6 | C57BL/6 | 2/3 (67%) | 22.1 ± 10.4 |
| | Synchimera | | | | |
| 4 | KB5 CBA/J | C57BL/6 | BALB/c | 5/5 (100%) | 27.1 ± 5.9 |
| | Synchimera | | | | |
| 5 | KB5 CBA/J | BALB/c | C57BL/6 | 0/4 (0%)^{a} | <0.5 |
| | Synchimera | | | | |

Reduction of High Numbers of Host Alloreactive CD8⁺ T Cells is Required for Bone Marrow Engraftment in KB5 Synchimeras

To determine the role of the spleen cell transfusion in facilitating subsequent engraftment of bone marrow cells, KB5 synchimeras were used. In addition to circulating their normal complement of alloreactive T cells, these mice also circulate large numbers (6-8%) of DES⁺CD8⁺ alloreactive (anti-H2-K^{b}) T cells (Iwakoshi et al., J. Immunol., 164:512-521, 2000). Using a standard protocol, which is known to delete DES⁺CD8⁺ peripheral T cells (Iwakoshi et al., J. Immunol., 164:512-521, 2000), it was observed that 2 of 3 KB5 synchimeras given a C57BL/6 DST, anti-CD154 mAb, and C57BL/6 bone marrow cells became chimeric (Table 5, Group 3). Confirming the results obtained in normal CBA/J mice (Table 5, Group 1), all KB5 synchimeras treated with C57BL/6 spleen cell transfusion plus anti-CD154 mAb and then given BALB/c (H2^{d}) bone arrow became chimeric (Table 5, Group 4). When the MHCs of the transfusion and bone marrow donors were reversed, in contrast, no KB5 synchimeric mice became allochimeric when treated with BALB/c spleen cell transfusion plus anti-CD154 mAb, and then given C57BL/6 (H2^{b}) bone marrow cells (Table 5, Group 5, p<0.01 vs. Group 4). This result suggests that, in the presence of large numbers of allospecific T cells in the recipient, as in the synchimera with large numbers of anti-H2-K^{b} T cells, the transfusion may need to be matched to that allospecificity.

### Example 6

### Host CD8⁺ Cell Deletion is Not Sufficient for Optimal Engraftment of Allogeneic Bone Marrow Cells

Previously, it has been shown that, in part, the role of DST in costimulation blockade protocols for peripheral tolerance induction is to enhance the deletion of host alloreactive CD8⁺ T cells (Iwakoshi et al., J. Immunol., 164:512-521, 2000; and Iwakoshi et al., J. Immunol., 167:6623-6630, 2001). The phenotyping analyses of KB5 synchimeras in which hematopoietic chimerism was generated successfully suggest that deletion of both peripheral host alloreactive CD8⁺ T cells and host alloreactive thymocytes is required. Next, the hypothesis that host CD8⁺ T cell deletion is required but not sufficient for establishing hematopoietic chimerism was tested by replacing the DST in the protocol described herein with a depleting anti-CD8 mAb. Briefly, BALB/c mice were randomized and injected with 50×10⁶ C57BL/6 bone marrow cells on day 0. All mice were injected intraperitoneally with 4 doses of 0.5 mg anti-CD154 mAb on days −7, −4, 0, +3 relative to bone marrow transplantation. In the groups indicated in Table 6, anti-CD8 (0.5 mg/dose), anti-CD4 (0.5 mg/dose), or anti-CTLA4 (0.075 mg/dose) mAb was injected intraperitoneally on days −7, −6, and −5 relative to bone marrow transplantation. Anti-CD122 mAb (1 mg/dose) was injected intraperitoneally on days −8 and −1 relative to bone marrow transplantation. Anti-CD25 mAb (0.25 mg/dose) was injected intraperitoneally on day −1. Mice in Groups 3, 4, and 5 received a single donor specific transfusion consisting of 10⁷ C57BL/6 spleen cells on day −7 relative to bone marrow cell transplantation.

The frequency of chimerism in BALB/c recipients treated with anti-CD8 mAb and anti-CD154 mAb before transplantation of C57BL/6 bone marrow was much lower (22% Table 6, Group 1) than in recipients treated with anti-CD154 mAb and DST (Table 1, Group 1, p <0.001). These results suggest that the role of DST in facilitating engraftment of allogeneic bone marrow cells involves mechanisms in addition to the deletion of host alloreactive CD8⁺ T cells.

TABLE 6
Frequency of chimerism in BALB/c Recipients of C57BL/6 Bone Marrow

| | | | | | | Percentage of |
|---|---|---|---|---|---|---|
| | | | | | Frequency | Donor Origin |
| | | | Bone | | of Hem. | PBMC in |
| | | DST | Marrow | Recipient | Chimerism | Chimeric |
| Group | Host | Donor | Donor | Treatment | (%) | Mice (%) |
| 1 | BALB/c | None | C57BL/6 | Anti-CD8 | 2/9 (22%)^{a} | 6.3, 4.8 |
| | | | | mAb | | |
| 2 | BALB/c | None | C57BL/6 | Anti- | 9/9 (100%)^{b} | 6.8 ± 2.4 |
| | | | | CD122 | | |
| | | | | mAb | | |
| 3 | BALB/c | C57BL/6 | C57BL/6 | Anti- | 2/9 (22%)^{c} | 5.5, 2.0 |
| | | | | CTLA4 | | |
| | | | | mAb | | |
| 4 | BALB/c | C57BL/6 | C57BL/6 | Anti-CD4 | 6/9 (67%)^{b} | 2.7 ± 1.2^{b} |
| | | | | mAb | | |
| 5 | BALB/c | C57BL/6 | C57BL/6 | Anti-CD25 | 8/9 (89%)^{b} | 6.0 ± 1.6 |
| | | | | mAb | | |

^{a}p < 0.005 vs. Group 2 and Table 1, Group 1.
^{b}p = N.S. vs. Table 1, Group 1.
^{c}p < 0.001 vs. Table 1, Group 1.

### Example 7

### Combined Treatment with Anti-CD154 mAb and Anti-CD122 mAb Leads to Hematopoietic Chimerism in BALB/c Recipients of C57BL/6 Bone Marrow

NK cells, which are CD122⁺, are known to be important in the rejection of allogeneic bone marrow (Yu et al., Ann. Rev. Immunol 10:189-213, 1992; Murphy et al., J. Natl. Cancer Inst. 85:1475-1482, 1993; Murphy et al., J. Exp. Med., 165:1212-1217, 1987; and Cudkowicz and Bennett, J. Exp. Med., 134:83-102, 1971). CD122 is expressed on most NK cells, activated macrophages, and a subset of activated CD8⁺ T cells and anti-CD122 mAb has been shown to delete NK cell activity in vivo (Tanaka et al., Immunol., 147:2222-2228, 1991; Tanaka et al., J. Exp. Med., 178:1103-1107, 1993; and Ehl et al., J. Immunol. Methods, 199:149-153, 1996). To begin to investigate the role of NK cell depletion in allogeneic bone marrow transplantation in mice treated with costimulation blockade, BALB/c mice were given anti-CD154 mAb, anti-CD122 mAb and 50×10⁶ C57BL/6 bone marrow cells. Surprisingly, hematopoietic chimerism was established in 100% of these recipients (9/9, Table 6, Group 2). This rate of successful engraftment is comparable to that achieved using DST in place of anti-CD122 mAb (89%, Table 1, Group 1, p=N.S.) and significantly greater than that achieved using anti-CD8 mAb in place of DST (Table 6, Group 1, p<0.01).

### Example 8

### Interventions that Abrogate Peripheral Tolerance Induction Impair Engraftment of Bone Marrow in Mice treated with Costimulation Blockade

Previously, it has been shown that injection of anti-CTLA4 mAb at the time of peripheral tolerance induction with DST and anti-CD154 mAb prevents deletion of alloreactive CD8⁺ T cells and shortens skin allograft survival (Markees et al., J. Clin. Invest., 101:2446-2455, 1998; Iwakoshi et al., J. Immunol., 164:512-521, 2000). Treatment with anti-CD4 mAb at the time of tolerance induction also shortens skin allograft survival (Markees et al., J. Clin. Invest., 101:2446-2455, 1998). Therefore, the hypothesis that these interventions would also interfere with the generation of hematopoietic chimerism in bone marrow recipients treated with DST and anti-CD154 mAb was evaluated. As shown in Table 6 (Group 4), treatment with anti-CD4 mAb had little effect, and two thirds of recipients became chimeric, albeit with a level of chimerism that was quite low (˜2.7%, p <0.001 vs. Table 1, Group 1). Similarly, in a cohort of recipients treated with an anti-CD25 mAb known to delete CD4⁺CD25⁺ regulatory T cells, nearly all (89%, N=9) became chimeric (Table 6, Group 5). In these mice the level of chimerism (6.0%) was greater than in the anti-CD4 treated mice (p<0.005), but not as high as in recipients treated with only DST and anti-CD154 mAb (˜9%, Table 1, Group 1, p<0.025). Only in the case of treatment with anti-CTLA4 mAb was there a significant reduction in the percentage of mice that became chimeric (22%, Table 6, Group 3). These results suggest that the mechanism by which the combination of DST plus anti-CD154 mAb generates peripheral transplantation tolerance is distinct but overlaps with the mechanism by which it generates hematopoietic chimerism.

### Example 9

### Induction of Chimerism and Allogeneic Skin Allograft Survival After Simultaneous Bone Marrow and Skin Graft Transplantation in the Absence of Irradiation

To determine whether the bone marrow transfusion and the tissue or organ transplantation could be performed simultaneously, two groups of BALB/c (H2^{d}) mice (N=10 in each group) were treated with a donor-specific transfusion consisting of 10⁷ C57BL/6 spleen cells on day −7 relative to transplantation of C57BL/6 skin allografts on day 0. They also received anti-CD154 mAb at a dose of 0.5 mg intraperitoneally on days −7, 4, 0, +3 relative to skin grafting (groups 1, 2, and 3). One group was also injected with C57BL/6 (H2^{b}) bone marrow cells (50×10⁶) on day 0 (groups 1 and 2). The percentage of donor-origin peripheral blood mononuclear cells (H2^{b+}; PBMC) was measured by flow cytometry in all mice 24 and 105 days after bone marrow transplantation. Two mice that were given C57BL/6 bone marrow and demonstrated <0.5% donor-origin mononuclear cells in the blood were considered non-chimeric (these mice were segregated for analysis purposes in group 2). In chimeric mice, the percentage of donor-origin mononuclear cells in the blood at day 57 ranged from 1.47 to 9.45% (mean=5.79+2.64%, n=8). Values indicated as “greater than” indicate an intact graft at the time the animal was removed from the study. a: p<0.001 group 1 vs. 3.

The results of these experiments, shown in Table 7, demonstrate that the bone marrow transfusion and the tissue or organ transplantation can successfully be performed simultaneously.

TABLE 7
Chimerism and Allogeneic Skin Allograft Survival After Simultaneous
Bone Marrow and Skin Graft Transplantation in the Absence of Irradiation

| | | | | | | | Skin |
|---|---|---|---|---|---|---|---|
| | | | Bone | | Skin | | Allograft |
| | | DST | Marrow | | Allograft | MST | Survival |
| Group | Host | Donor | Donor | Chimeric | Donor | (Days) | (Days) |
| 1 | BALB/c | C57BL/6 | C57BL/6 | Yes | C57BL/6 | >112^{a} | >73, 81, |
| | | | | (8/10) | | | 104, |
| | | | | | | | >112 × 5^{a} |
| 2 | BALB/c | C57BL/6 | C57BL/6 | No | C57BL/6 | 15 | 8, 22 |
| | | | | (2/10) | | | |
| 3 | BALB/c | C57BL/6 | None | No | C57BL/6 | 49 | 39, 39, 41, |
| | | | | | | | 43, 49, 49, |
| | | | | | | | 54, 59, 79, |
| | | | | | | | 97 |

### Example 10

### Allogeneic Chimerism Established in Outbred CF1 Mice in the Absence of Irradiation: Enhancement of Chimerism by Injection of Anti-CD122 Antibody

To determine whether the methods described herein can be used on non-inbred mammals, CF1 outbred mice (Charles River Laboratories, Inc., Wilmington, Mass.; Groups 1 and 2) and inbred BALB/c mice (Group 3) were treated with a donor-specific priming transfusion consisting of 10⁷ C57BL/6 spleen cells on day −7 relative to transplantation of 50×10⁶ C57BL/6-GFP-positive bone marrow cells on day 0. The mice also received anti-CD154 mAb at a dose of 0.5 mg intraperitoneally on days −7, −4, 0, +3 relative to bone marrow cell injection. Mice in Group 2 were also injected with 1 mg of anti-CD122 mAb day −8 and −1 relative to bone marrow injection on day 0. The percentage of donor-origin peripheral blood mononuclear cells (H2^{b+}; PBMC) was measured by flow cytometry analysis of circulating GFP⁺ cells in all mice 56 days after bone marrow transplantation. Chimerism levels represent the mean±1 s.d. of 5 mice per group. a: p<0.05 vs. group 1. The data, shown in Table 8, demonstrate that the methods described herein can be used on non-inbred mammals.

TABLE 8
Allogeneic Chimerism Established in Outbred CF1 Mice in the Absence of
Irradiation: Enhancement of Chimerism by Injection of Anti-CD122 Antibody

| | | | | | | | Chimerism |
|---|---|---|---|---|---|---|---|
| | | | | | | | (% GFP⁺ |
| | | | | | | | Peripheral |
| | | | anti- | Bone | | Anti- | Blood |
| | | DST | CD154 | Marrow | | CD122 | Cells; |
| Group | Host | Donor | mAb | Donor | Irradiation | Antibody | Mean ± s.d.) |
| 1 | CF1 | C57BL/6 | Yes | C57BL/6- | No | No | 1.04 ± 1.33 |
| | | | | GFP | | | |
| 2 | CF1 | C57BL/6 | Yes | C57BL/6- | No | Yes | 11.4 ± 9.17^{a} |
| | | | | GFP | | | |
| 3 | BALB/c | C57BL/6 | Yes | C57BL/6- | No | No | 9.87 ± 2.47^{a} |
| | | | | GFP | | | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of inducing hematopoietic chimerism and central tolerance in a transplant recipient, the method comprising:
administering to the recipient a priming transfusion comprising an allogeneic or xenogeneic cell, wherein the cell comprises on its surface a ligand that interacts with a receptor on a surface of a recipient T cell that mediates contact-dependent helper-effector function;
administering to the recipient one or more doses of a receptor antagonist that inhibits interaction of the ligand with the receptor; and
administering to the recipient a hematopoietic stem cell transplant; thereby inducing hematopoietic chimerism and central tolerance in the recipient.

2. The method of claim 1, wherein the allogeneic or xenogeneic cell expresses a donor or third-party antigen.

3. The method of claim 1, wherein the receptor antagonist is a CD154 antagonist.

4. The method of claim 3, wherein the CD154 antagonist is an anti-CD154 antibody or antigen-binding fragment thereof.

5. The method of claim 1, wherein the hematopoietic stem cell transplant is a bone marrow transplant.

6. The method of claim 5, wherein the bone marrow transplant is a low-dose bone marrow transplant.

7. The method of claim 1, further comprising administering to the recipient one or more doses of a CD122 antagonist.

8. The method of claim 7, wherein the CD122 antagonist is an anti-CD122 antibody or antigen-binding fragment thereof.

9. The method of claim 1, wherein the method does not include host myeloablative conditioning.

10. The method of claim 1, wherein the method includes minimal myeloablative conditioning.

11. The method of claim 1, wherein the priming transfusion and receptor antagonist are administered at least three days before the hematopoietic stem cell transplant.

12. The method of claim 1, wherein the priming transfusion and receptor antagonist are administered at least seven days before the hematopoietic stem cell transplant.

13. The method of claim 1, wherein the priming transfusion and receptor antagonist are administered concurrently.

14. The method of claim 1, wherein the receptor antagonist is administered at one or more of before, concurrently with, or after the priming transfusion.

15. The method of claim 1, further comprising implanting into the recipient a tissue or organ.

16. The method of claim 15, wherein the tissue or organ is implanted concurrently with or after administering the hematopoietic stem cell transplant.

17. A method of inducing hematopoietic chimerism and central tolerance in a transplant recipient, the method comprising:
administering to the recipient one or more doses of a CD122 antagonist;
administering to the recipient one or more doses of a CD154 antagonist;
administering to the recipient a hematopoietic stem cell transplant; and
thereby inducing hematopoietic chimerism and central tolerance in the recipient.

18. The method of claim 17, further comprising administering to the recipient a priming transfusion comprising an allogeneic or xenogeneic cell, wherein the cell comprises on its surface a ligand that interacts with a receptor on a surface of a recipient T cell that mediates contact-dependent helper-effector function.

19. The method of claim 18, wherein the allogeneic or xenogeneic cell expresses a donor or third-party antigen.

20. The method of claim 17, wherein the CD154 antagonist is an anti-CD154 antibody or antigen-binding fragment thereof.

21. The method of claim 17, wherein the CD154 antagonist is an anti-CD40 antibody or antigen-binding fragment thereof.

22. The method of claim 17, wherein the hematopoietic stem cell transplant is a bone marrow transplant.

23. The method of claim 17, wherein the bone marrow transplant is a low-dose bone marrow transplant.

24. The method of claim 17, wherein the CD122 antagonist is an anti-CD122 antibody or antigen-binding fragment thereof.

25. The method of claim 17, wherein the method does not include host myeloablative conditioning.

26. The method of claim 17, wherein the method includes minimal myeloablative conditioning.

27. The method of claim 17, wherein the CD122 antagonist and receptor antagonist are administered before the hematopoietic stem cell transplant.

28. The method of claim 17, wherein the CD122 antagonist and receptor antagonist are administered concurrently.

29. The method of claim 17, comprising administering at least two doses of the CD122 antagonist.

30. The method of claim 17, further comprising implanting into the recipient a tissue or organ.

31. The method of claim 31, wherein the tissue or organ is implanted concurrently with or after the hematopoietic stem cell transplant.

32. A kit comprising a CD122 antagonist and a CD154 antagonist, and instructions for use in inducing hematopoietic chimerism and central tolerance in a transplant recipient.

33. A therapeutic composition comprising a CD122 antagonist, a CD154 antagonist, and a pharmaceutically acceptable carrier.
